(19) United States
(12) Patent Application Publication
(10) Pub. No.: US 2024/0175066 A1
(43) Pub. Date: May 30, 2024

(54) MODIFIED HOST CELLS FOR HIGH EFFICIENCY PRODUCTION OF VANILLIN

(71) Applicant: AMYRIS , INC., Emeryville, CA (US)

(72) Inventors: Nathaniel KREFMAN, Emeryville, CA (US); Lauren RAETZ, Emeryville, CA (US); Elizabeth ZNAMERROSKI, Emeryville, CA (US)

(21) Appl. No.: 18/551,182

(22) PCT Filed: Mar. 18, 2022
(86) PCT No.: PCT/US2022/021017

§ 371 Date: Sep. 18, 2023

Related U.S. Application Data
(60) Provisional application No. 63/163,223, filed on Mar. 19, 2021.

(51) Int. Cl. C12P 19/44; C12N 1/18; C12N 9/02; C12N 9/10; C12N 9/12; C12N 9/88; C12N 15/52; C12P 7/24

(52) U.S. Cl. CPC C12P 19/44; C12N 1/185; C12N 9/0008; C12N 9/1011; C12N 9/1051; C12N 9/1085; C12N 9/1288; C12N 9/88; C12N 15/52; C12P 7/24; C12Y 102/0103; C12Y 201/01114; C12Y 204/01126; C12Y 205/01054; C12Y 207/08007; C12Y 402/0101; C12Y 402/01118

(57) ABSTRACT

Provided herein are genetically modified host cells. compositions. and methods for improved production of vanillin and/or glucovanillin. The host cells. compositions. and methods described herein provide an efficient route for the heterologous production of vanillin and/or glucovanillin and any compound that can be synthesized or biosynthesized from either or both.

### CROSS-REFERENCE TO RELATED APPLICATIONS

The present application claims the benefit of U.S. provisional application no. 63/163,223, filed Mar. 19. 2021, the content of which is hereby incorporated by reference in its entirety.

### INCORPORATION BY REFERENCE OF SEQUENCE LISTING

This application contains a sequence listing entitled “107345_00892_SEQLISTING.txt,” being submitted herein in ASCII format via EFS-Web, which was created on Mar. 18, 2022 and is 39,101 bytes in size.

### FIELD OF THE INVENTION

The present disclosure relates to particular genetic modifications, host cells comprising the same, and methods of their use for the production of vanillin and/or glucovanillin and any compound that can be synthesized or biosynthesized from either or both.

### BACKGROUND

Vanillin is the largest-volume flavor ingredient in the world. Only about 1% of the vanilla flavor ingredient supply comes from vanilla extract from the vanilla orchid. There is strong demand, insufficient supply, and a high price for “natural” vanillin. An alternative, low cost, high-volume source of “natural” vanillin would be a lucrative addition to the flavorings market. Vanillin produced de novo through fermentation of sugar by yeast has the potential to generate “natural” vanillin at a lower cost than alternatives currently in the market.

There are several approaches that are being used to generate “natural” vanillin by bioconversion from natural precursors, including precursors other than glucose. One path is bioconversion of ferulic acid which is found abundantly in certain parts of certain plants. Microorganisms have been identified which catabolize ferulic acid by a pathway which generates vanillin as an intermediate. These microorganisms can be engineered to reduce further catabolismof vanillin to unwanted side products to optimize vanillin production. Gallage et al., Molecular Plant. 8: 40-57 (2015). In a similar approach, the more cost-effective substrate eugenol can be catabolized by microorganisms to ferulic acid and further to vanillin. Gallage et al.

There is no known microorganism that can natively convert glucose to vanillin. Gallage et al. In 1998, an enzymatic route from glucose to vanillin was developed which converts a natively produced metabolite 3-dehydroshikimate into vanillin with three additional enzymatic steps: 1.) dehydration to produce protocatechuic acid (3,4-dihydroxy benzoic acid) 2.) O-methylation of the 3-hydroxyl group, and 3.) reduction of the carboxylic acid to an aldehyde. Li and Frost, J. Am. Chem. Soc., 120: 10545-10546 (1998). This process was demonstrated by producing vanillic acid (steps 1 and 2) in E. coli by expression of heterologous enzymes catalyzing 3-DHS dehydratase (AroZ) and catechol-O-methyltransferase (COMT). An enzymatic conversion using an aromatic carboxylic acid reductase (ACAR) purified from fungi was used to convert vanillic acid to vanillin in vitro.

Hansen et al. demonstrated de novo biosynthesis of vanillin from glucose in a single recombinant organism, Saccharomyces cerevisiae, by expressing the above enzymes in combination with a heterologous PPTase which was identified to be necessary to activate the ACAR enzyme in this organism. Hansen et al., Appl. Environ. Microbiol. 75:2765-2774 (2009). In addition they expressed a UDP-glucosyltransferase to convert the toxic vanillin product into the far less toxic glucovanillin.

A number of other modifications have been reported to improve the efficiency of vanillin biosynthesis in yeast. In order to improve titer of glucovanillin, Hansen et al. demonstrated that it was important to reduce endogenous reductase activity through the deletion of native reductases (i.e. ADH6) to reduce conversion of vanillin to vanillyl alcohol. In order to mitigate loss of carbon to the undesired isomer, isovanillin (produced by methylation of the 4-OH instead of 3-OH), the human variant Hs. COMT was used as a starting point for enzyme evolution. US 2014/0245496: WO 2015/121379. Mutants were obtained which were highly specific for the correct vanillin isomer. In order to increase flux to PCA and reduce flux to shikimate pathway metabolites, a mutant version of Aro1 was generated, annotated as mutant AROM which contains a mutation in the E domain and reduces activity of this reaction that uses 3-DHS as a substrate to make shikimate.

Further genetic modifications that can provide low cost, high-volume sources of “natural” vanillin would be a significant addition to the flavorings market.

### SUMMARY OF THE INVENTION

Provided herein are genetically modified host cells, compositions, and methods for the improved production of vanillin and/or glucovanillin. These compositions and methods are based in part on the deletion or modified production of certain gene products including a glycerol dehydrogenase, GCY1, and homologs thereof, in host cells that are optionally further genetically modified to produce vanillin and/or glucovanillin. While not intending to be bound by any particular theory of operation, the examples herein demonstrate that cells lacking GCY1 or encoding a modified GCY1 substantially reduce conversion of the products of vanillin and glucovanillin synthesis into byproducts such as vanillyl alcohol and glucovanillyl alcohol, thereby maintaining yields of vanillin and glucovanillin.

In one aspect, provided herein are genetically modified host cells and methods of their use for the production of vanillin or glucovanillin. In certain embodiments, provided herein are genetically modified host cells capable of producing vanillin or glucovanillin with lower levels of the byproducts vanillyl alcohol and glucovanillyl alcohol, where the host cell has a modified or absent glycerol dehydrogenase. In particular embodiments, the genetically modified host cell expresses elevated levels of vanillin or glucovanillin and lower levels of the byproducts vanillyl alcohol and/or glucovanillyl alcohol. Useful enzymes are described herein.

In another aspect, provided herein is a method for producing vanillin or glucovanillin involving culturing a population of the host cells of the invention in a medium with a carbon source under conditions suitable for making vanillin or glucovanillin to yield a culture broth; and recovering the vanillin or glucovanillin from the culture broth.

In a further aspect. provided herein is vanillin produced by a method provided herein.

### BRIEF DESCRIPTION OF THE FIGURES

FIG. 1 is a schematic showing an enzymatic pathway from glucose to vanillin and glucovanillin including the undesired production of vanillyl alcohol by the native GCY inside dotted box.

FIG. 2 is a graph providing titers of g/L vanillin and g/L vanillyl alcohol for a 96-well plate experiment using a vanillin producing strain with the GCY1 intact (Parent) and the ORF of the GCY1 deleted (GCY1\hat{ }), the GCY1 A53T point mutation (GCY1 A53T), or the 100 base pairs immediately upstream of the start codon (ATG) replaced with the pG2MAL promoter (pG2MAL>GCY1). In all cases, samples are treated with a commercially available beta-glucosidase to convert glucovanillin into vanillin and gluco-vanillyl alcohol into vanillyl alcohol before quantification. Note that in strains labeled GCY1\hat{ }, GCY1 A53T, or pG2MAL>GCYI the amount of vanillyl alcohol produced is below the linear range of the assay (less than 0.12 g/L).

FIG. 3A is a graph providing Cumulative Yield (weight %; vanillin) and Cumulative Productivity (g/L/h: vanillin) for a 7 day fermentation using a vanillin producing strain with either the wild type GCY1 intact (Parent: black), the ORF of the GCY1 gene deleted (GCY1\hat{ }; dark grey), or the 100 base pairs immediately upstream of the start codon (ATG) replaced with the pG2MAL promoter (pG2MAL>GCY1: light grey). “Cumulative” indicates the value for the interval from time zero to the indicated time.

FIG. 3B is a graph providing titers (g/L) of vanillyl alcohol for a 7 day fermentation using vanillin producing strain with either the wild type GCY1 intact (Parent: black), the ORF of the GCY1 gene deleted (GCY1\hat{ }: dark grey), or the 100 base pairs immediately upstream of the Start codon replaced with the pG2MAL promoter (pG2MAL>GCY1: light grey).

FIG. 4 provides a graph comparing titers of g/L vanillin and g/L vanillyl alcohol for a 96-well plate experiment using a vanillin producing strain with the GCY1 and SKY1 intact (Parent), the ORF of the GCY1 deleted (GCY1\hat{ }), or the ORF of the GCY1 deleted and the ORF of the SKY1 deleted (GCY1\hat{ } SKY1\hat{ }).

FIG. 5 provides the GCY1 transcript sequence as provided in NM_001183539 (SEQ ID NO:1).

FIG. 6 provides the GCY1 amino acid sequence as disclosed in NP_014763 (SEQ ID NO:2).

### DETAILED DESCRIPTION OF THE EMBODIMENTS

### Terminology

As used herein, the term “about” refers to a reasonable range about a value as determined by the practitioner of skill. In certain embodiments, the term about refers to ±one, two, or three standard deviations. In certain embodiments, the term about refers to ±5%, 10%, 20%, or 25%. In certain embodiments, the term about refers to ±0.1, 0.2, or 0.3 logarithmic units, e.g. pH units.

As used herein, the term “heterologous” refers to what is not normally found in a cell in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

On the other hand, the term “native” or “endogenous” as used herein with reference to molecules, and in particular enzymes and nucleic acids, indicates molecules that are expressed in the organism in which they originated or are found in nature. It is understood that expression of native enzymes or polynucleotides may be modified in recombinant microorganisms. In particular embodiments, codon optimized genes express native enzymes.

As used herein, the term “heterologous nucleic acid expression cassette” refers to a nucleic acid sequence that comprises a coding sequence operably linked to one or more regulatory elements sufficient to expresses the coding sequence in a host cell. Non-limiting examples of regulatory elements include promoters, enhancers, silencers, terminators, and poly-A signals.

As used herein, the terms “homolog of fatty aldehyde dehydrogenase” and “HFD1” or “Hfd1” refer to an encoding nucleic acid and a dehydrogenase involved in ubiquinone and sphingolipid metabolism capable of converting 4-hydroxy benzaldehyde into 4-hydroxy benzoate for ubiquinone anabolism and/or hexadecenal to hexadecenoic acid in sphingosine 1-phosphate catabolismin certain embodiments, its EC number is 1.2.1.3. In certain embodiments, its sequence is according to NCBI Reference Sequence NP_013828 or S. cerevisiae YMR110C.

As used herein, the terms “NADPH-dependent medium chain alcohol dehydrogenase” and “ADH6” or “Adh6” refer to an encoding nucleic acid and an alcohol dehydrogenase. In certain embodiments, its EC number is 1.1.1.2. In certain embodiments, its sequence is according to GenBank locus CAA90836 or S. cerevisiae YMR318C.

As used herein, the terms “3-methylbutanal reductase” and “NADPH-dependent methylglyoxal reductase” and “GRE2” or “Gre2” refer to an encoding nucleic acid and a 3-methylbutanal reductase and NADPH-dependent methylglyoxal reductase. In certain embodiments, its EC number is 1.1.1.265 or 1.1.1.283. In certain embodiments, its sequence is according to NCBI reference sequence NP_014490 or S. cerevisiae YOL151W.

As used herein, the term “YGL039W” refers to an encoding nucleic acid and an aldehyde reductase. Its systematic name is YGL039W. In certain embodiments, its sequence is according to GenBank reference Z72561.

As used herein, the term “YOR120W” refers to an encoding nucleic acid and an glycerol dehydrogenase. In some embodiments, its sequence is according to GenBank reference NM_001183539 (SEQ ID NO:1) NP_014763

As used herein, the terms “dihydrofolate reductase” and “DHFR” refer to an encoding nucleic acid and a dihydrofolate reductase. In certain embodiments, its EC number is 1.5.1.3. In certain embodiments, DHFR is from Mus musculus. In certain embodiments. the DHFR sequence is according to NCBI reference sequence NP_034179.

As used herein, the terms “3-dehydroquinate synthase” and “AroB” refer to an encoding nucleic acid and a 3-dehydroquinate synthase. In certain embodiments, its EC number is 4.2.3.4. In certain embodiments, AroB is from E. coli. In certain embodiments, the AroB sequence is according to UniProtKB P07639.

As used herein, the terms “3-dehydroquinate dehydratase” and “AroD” refer to an encoding nucleic acid and a 3-dehydroquinate dehydratase. In certain embodiments, its EC number is 4.2.1.10. In certain embodiments, AroD is from E. coli. In certain embodiments, the AroD sequence is according to UniProtKB P05194.

As used herein, the terms “phospho-2-dehydro-3-deoxyheptonate aldolase. Tyr-sensitive” and “AroF” refer to an encoding nucleic acid and a phospho-2-dehydro-3-deoxyheptonate aldolase. In certain embodiments, its EC number is 2.5.1.54. In certain embodiments, AroF is from E. coli. In certain embodiments, the AroF sequence is according to UniProtKB P00888. In certain embodiments, the AroF is feedback resistant (J. Bacteriol. November 1990 172:6581-6584).

As used herein, the terms “3-dehydroshikimate dehydratase” and “AroZ” refer to an encoding nucleic acid and a 3-dehydroshikimate dehydratase. In certain embodiments, its EC number is 4.2.1.118. In certain embodiments, AroZ is from Podospora pauciseta. In certain embodiments, the AroZ sequence is according to Hansen et al., Appl Environ Microbiol. 2009 (May) 75(9):2765-74.

As used herein, the terms “phosphopantetheinyl transferase” and “PPTASE” refer to an encoding nucleic acid and a phosphopantetheinyl transferase. In certain embodiments, its EC number is 2.7.8.7. In certain embodiments, PPTASE is from Corynebacterium glutamicum. In certain embodiments, the PPTASE sequence is according to UniProtKB Q8NP45.

As used herein, the terms “O-methyltransferase” and “OMT” refer to an encoding nucleic acid and an O-methyltransferase. It can be from a variety of host cells. In certain embodiments, for example, the OMT is from yeast and has an EC number 2.1.1.6. In some embodiments, the OMT is from Brachypodium distachyon and has an EC number 2.1.1.114. In certain embodiments, OMT is from Saccharomyces cerevisiae.

As used herein, the terms “aromatic carboxylic acid reductase” and “ACAR” refer to an encoding nucleic acid and an aromatic carboxylic acid reductase. In certain embodiments, its EC number is 1.2.1.30. In certain embodiments, ACAR is from Nocardia iowensis. In certain embodiments, the ACAR sequence is according to UniProtKB Q6RKB1.

As used herein, the terms “eugenol alcohol oxidase” and “EAO” refer to an encoding nucleic acid and a eugenol alcohol oxidase. In certain embodiments, EAO is from Rhodococcus jostii. In certain embodiments, the EAO sequence is according to UniProtKB QOSBK1.

As used herein, the terms “UDP-glycosyltransferase” and “UGT” refer to an encoding nucleic acid and a UDP-glycosyltransferase. In certain embodiments, its EC number is 2.4.1.126. In certain embodiments, the UGT is from Arabidopsis thaliana. In certain embodiments, the UGT is A. thaliana UGT72E2. In certain embodiments, the UGT sequence is according to UniProtKB Q9LVR1.

As used herein, the term “serine/threonine protein kinase” and “SKY1” refer to an encoding nucleic acid and a serine/threonine protein kinase. In certain embodiments, its EC number is 2.7.11.1. In certain embodiments, the Sky1 is from Saccharomyces cerevisiae, e.g., strain 204508/S288c. In certain embodiments, the SKY1 sequence is according to FIG. 5. In certain embodiments, the Skyl peptide sequence is according to UniProtKB Q03656.

As used herein, the term “glycerol dehydrogenase” and “GCY1” refer to an encoding nucleic acid and a glycerol dehydrogenase. In certain embodiments, its EC number is 1.1.1.156. In certain embodiments, the GCY1 is from Saccharomyces cerevisiae, e.g., strain (CEN.PK). In certain embodiments the GCY1 sequence is according to FIG. 5. In certain embodiments, the Gcyl peptide is according to UniProtKB P14065.

As used herein, the term “parent cell” refers to a cell that has an identical genetic background as a genetically modified host cell disclosed herein except that it does not comprise one or more particular genetic modifications engineered into the modified host cell, for example, one or more modifications selected from the group consisting of: heterologous expression of an enzyme of a vanillin pathway, heterologous expression of an enzyme of a glucovanillin pathway: or heterologous expression of Gcy1, AroB. AroD, AroF, AroZ, PPTASE, or ACAR: or deletion of HFD1, ADH6, GRE2, or YGL039W.

As used herein, the term “naturally occurring” refers to what is found in nature. For example, gene product that is present in an organism that can be isolated from a source in nature and that has not been intentionally modified by a human in the laboratory is naturally occurring gene product. Conversely, as used herein, the term “non-naturally occurring” refers to what is not found in nature but is created by human intervention. In certain embodiments, naturally occurring genomic sequences are modified, e.g. codon optimized, for use in the organisms provided herein.

The term “medium” refers to a culture medium and/or fermentation medium.

The term “fermentation composition” refers to a composition which comprises genetically modified host cells and products or metabolites produced by the genetically modified host cells. An example of a fermentation composition is a whole cell broth, which can be the entire contents of a vessel (e.g., a flasks, plate, or fermentor), including cells, aqueous phase, and compounds produced from the genetically modified host cells.

As used herein, the term “production” generally refers to an amount of vanillin or a derivative thereof produced by a genetically modified host cell provided herein. Derivatives can include glucovanillin, vanillyl alcohol, and/or vanillic acid. In some embodiments, production is expressed as a yield of vanillin or glucovanillin by the host cell. In other embodiments, production is expressed as the productivity of the host cell in producing the vanillin or glucovanillin.

As used herein, the term “productivity” refers to production of a vanillin or a derivative thereof by a host cell, expressed as the amount of vanillin or glucovanillin produced (by weight) per amount of fermentation broth in which the host cell is cultured (by volume) over time (per hour). Derivatives can include glucovanillin, vanillyl alcohol, and/or vanillic acid.

As used herein, the term “yield” refers to production of a vanillin or a derivative thereof by a host cell, expressed as the amount of vanillin or glucovanillin produced per amount of carbon source consumed by the host cell, by weight. Derivatives can include glucovanillin, vanillyl alcohol, and/or vanillic acid.

As used herein, the term “titer” refers to production of a vanillin or a derivative thereof by a host cell, expressed as the amount of vanillin or glucovanillin or other derivative produced per volume of media. Derivatives can include glucovanillin, vanillyl alcohol, and/or vanillic acid.

As used herein, the term “an undetectable level” of a compound (e.g., vanillic acid, or other compounds) means a level of a compound that is too low to be measured and/or analyzed by a standard technique for measuring the compound. For instance, the term includes the level of a compound that is not detectable by the typical analytical methods known in the art.

The term “vanillin” refers to the compound vanillin, including any stereoisomer of vanillin. The chemical name of vanillin is 4-hydroxy-3-methoxybenzaldehyde. In particular embodiments, the term refers to the compound according to the following structure:

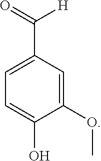

The term “vanillyl alcohol” refers to the compound vanillyl alcohol, including any stereoisomer of vanillyl alcohol. The chemical name of vanillyl alcohol is 4-(hydroxymethyl)-2-methoxyphenol. In particular embodiments, the term refers to the compound according to the following structure:

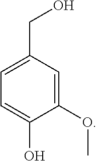

The term “vanillic acid” refers to the compound vanillic acid, including any stereoisomer of vanillic acid. The chemical name of vanillic acid is 4-hydroxy-3-methoxybenzoic acid. In particular embodiments, the term refers to the compound according to the following structure:

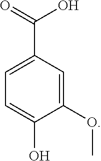

The term “glucovanillin” refers to the compound glucovanillin, including any stereoisomer of glucovanillin. The chemical name of glucovanillin is 3-methoxy-4-[(2S,3R,4S,5S,6R)-3,4,5-trihydroxy-6-(hydroxymethyl)oxan-2-yl]oxybenzaldehyde. In particular embodiments, the term refers to the compound according to the following structure:

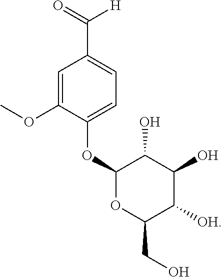 [SMILES: COc1cc(C=O)ccc1O[C@@H]1O[C@H](CO)[C@@H](O)[C@H](O)[C@H]1O]

The term “glucovanillyl alcohol” refers to the compound glucovanillyl alcohol, including any stereoisomer of glucovanillyl alcohol. The chemical name of glucovanillyl alcohol is vanillyl alcohol 4-0-beta-D-glucopyranoside or (2R,3S,4S,5R,6S)-2-(hydroxymethyl)-6-(4-(hydroxymethyl)-2-methoxyphenoxy)tetrahydro-2H-pyran-3,4,5-triol. In particular embodiments, the term refers to the compound according to the following structure:

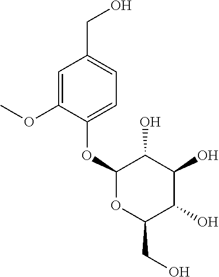 [SMILES: COc1cc(CO)ccc1O[C@@H]1O[C@H](CO)[C@@H](O)[C@H](O)[C@H]1O]

The term “protecatechuic acid” refers to the compound protecatechuic acid , including any stereoisomer of protecatechuic acid . The chemical name of protecatechuic acid is 3,4-dihydroxybenzoic acid. In particular embodiments, the term refers to the compound according to the following structure:

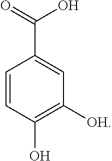 [SMILES: O=C(O)c1ccc(O)c(O)c1]

As used herein, the term “variant” refers to a polypeptide differing from a specifically recited “reference” polypeptide (e.g .. a wild-type sequence) by amino acid insertions, deletions, mutations, and/or substitutions, but retains an activity that is substantially similar to the reference polypeptide. In some embodiments, the variant is created by recombinant DNA techniques or by mutagenesis. In some embodiments, a variant polypeptide differs from its reference polypeptide by the substitution of one basic residue for another (i.e. Arg for Lys), the substitution of one hydrophobic residue for another (i.e. Leu for Ile), or the substitution of one aromatic residue for another (i.e. Phe for Tyr), etc. In some embodiments, variants include analogs wherein conservative substitutions resulting in a substantial structural analogy of the reference sequence are obtained. Examples of such conservative substitutions, without limitation, include glutamic acid for aspartic acid and vice-versa: glutamine for asparagine and vice-versa: serine for threonine and vice-versa: lysine for arginine and vice-versa: or any of isoleucine, valine or leucine for each other.

As used herein, the term “sequence identity” or “percent identity,” in the context or two or more nucleic acid or protein sequences. refers to two or more sequences or subsequences that are the same or have a specified percentage of amino acid residues or nucleotides that are the same. For example, the sequence can have a percent identity of at least 60%, at least 65%, at least 70%, at least 75%, at least 80%, at least 85%, at least 90%, at least 91% at least 92%, at least 93%, at least 94%, at least 95%, at least 96%, at least 97%, at least 98%. at least 99%, or higher identity over a specified region to a reference sequence when compared and aligned for maximum correspondence over a comparison window, or designated region as measured using a sequence comparison algorithm or by manual alignment and visual inspection. For example, percent of identity is determined by calculating the ratio of the number of identical nucleotides (or amino acid residues) in the sequence divided by the length of the total nucleotides (or amino acid residues) minus the lengths of any gaps.

For convenience, the extent of identity between two sequences can be ascertained using computer programs and mathematical algorithms known in the art. Such algorithms that calculate percent sequence identity generally account for sequence gaps and mismatches over the comparison region. Programs that compare and align sequences, like Clustal W (Thompson et al., (1994) Nucleic Acids Res., 22: 4673-4680), ALIGN (Myers et al., (1988) CABIOS, 4: 11-17), FASTA (Pearson et al., (1988) PNAS, 85:2444-2448: Pearson (1990), Methods Enzymol., 183: 63-98) and gapped BLAST (Altschul et al., (1997) Nucleic Acids Res., 25: 3389-3402) are useful for this purpose. The BLAST or BLAST 2.0 (Altschul et al., J. Mol. Biol. 215:403-10, 1990) is available from several sources, including the National Center for Biological Information (NCBI) and on the Internet, for use in connection with the sequence analysis programs BLASTP, BLASTN, BLASTX, TBLASTN, and TBLASTX. Additional information can be found at the NCBI web site.

In certain embodiments, the sequence alignments and percent identity calculations can be determined using the BLAST program using its standard, default parameters. For nucleotide sequence alignment and sequence identity calculations, the BLASTN program is used with its default parameters (Gap opening penalty=5, Gap extension penalty=2, Nucleic match=2, Nucleic mismatch=−3, Expectation value=10.0, Word size=11, Max matches in a query range=0). For polypeptide sequence alignment and sequence identity calculations, BLASTP program is used with its default parameters (Alignment matrix=BLOSUM62: Gap costs: Existence=11, Extension=1: Compositional adjustments-Conditional compositional score, matrix adjustment: Expectation value=10.0: Word size=6; Max matches in a query range=0). Alternatively, the following program and parameters can be used: Align Plus software of Clone Manager Suite. version 5 (Sci-Ed Software): DNA comparison: Global comparison, Standard Linear Scoring matrix, Mismatch penalty=2. Open gap penalty=4, Extend gap penalty=1. Amino acid comparison: Global comparison, BLOSUM 62 Scoring matrix. In the embodiments described herein, the sequence identity is calculated using BLASTN or BLASTP programs using their default parameters. In the embodiments described herein, the sequence alignment of two or more sequences are performed using Clustal W using the suggested default parameters (Dealign input sequences: no: Mbed-like clustering guide-tree: yes: Mbed-like clustering iteration: yes: number of combined iterations: default(0): Max guide tree iterations: default: Max HMM iterations: default: Order: input).

### Nucleic Acids, Expression Cassettes, and Host Cells

In one aspect, provided herein are nucleic acids, expression vectors, and host cells which express one or more enzymes useful for the production of vanillin and/or glucovanillin. In another aspect, provided herein are host cells comprising one or more deletions in genes wherein the one or more deletions are useful for the production of vanillin and/or glucovanillin. In a further aspect, provided herein are host cells that comprise one or more of the deletions and further comprise one or more of the enzymes. The enzymes and deletions are described in detail herein. In certain embodiments, the host cells can produce vanillin and/or glucovanillin from a carbon source in a culture medium. In certain embodiments, the host cells provide improved yield and/or productivity compared to a parent strain. In certain embodiments, the host cells provide byproducts, intermediates, and/or side products, e.g. vanillic acid, compared to a parent strain. Exemplary byproducts, intermediates, and/or side products include vanillic acid, vanillyl alcohol, glucovanillic acid, glucovanillyl alcohol, and protocatechuic aldehyde.

In certain embodiments, host cells according to the embodiments herein produce at least 5%, at least 10%, at least 15%, at least 20%, or at least 25% more total vanillin or glucovanillin compared to a parent strain. In certain embodiments, host cells according to the embodiments herein produce at least 5%, at least 10%, at least 15%, at least 20%, at least 25% more total vanillin compared to a parent strain. In certain embodiments, host cells according to the embodiments herein produce at least 5%, at least 10%, at least 15%, at least 20%, at least 25% more total glucovanillin compared to a parent strain. In certain embodiments, host cells according to the embodiments herein produce 2-fold, 3-fold, 4-fold, 5-fold. or 10-fold less vanillic acid compared to a parent strain. In certain embodiments. the percent increases are with respect to vanillin or glucovanillin titer (g/L). In certain embodiments, the percent increases are with respect to vanillin or glucovanillin yield (weight %). In certain embodiments, the percent increases are with respect to vanillin or glucovanillin productivity (g/L/h). In certain embodiments. the percent increases are with respect to vanillin or glucovanillin total mass produced (g). Those of skill will recognize that the total vanillin and/or glucovanillin produced can be measured as a sum of the actual compounds produced and any downstream compounds produced from the vanillin and/or glucovanillin, as shown in the Examples and Figures herein. In certain embodiments. host cells according to the embodiments herein produce increased vanillin and/or glucovanillin, and produce less vanillic acid, compared to a parent strain.

In particular embodiments, the host cell comprises reduction or elimination of expression and/or activity of GCY1 in the cell. In certain embodiments, the host cell further comprises one or more enzymatic pathways capable of making vanillin and/or glucovanillin, said pathways taken individually or together.

The glycerol dehydrogenase encoded by GCY1 is involved in an alternative pathway for glycerol catabolismused under microaerobic conditions. Overexpression of GCY1 enhanced vanillin tolerance in yeast strain BY4741. Gcy1p has been shown to have NADPH-dependent vanillin reductase activity, which can directly increase vanillin tolerance Liang et al., (2020) Microbial Biotechnology, doi: 10.1111/1751-7915.13643.

In some embodiments, expression of GCY1 in the host cell is reduced to less than 80%, 75%, 70%, 65%, 60%, 55%, 50%, 45%, 40%, 35%, 30%, 25%, 20%, 15%, 10%, 5% or 2% of wild type expression measured by any established techniques. In some embodiments. said reduction of activity is measured on a per cell mass basis.

In some embodiments, Gcy1p activity is reduced to less than 80%, 75%, 70%, 65%, 60%, 55%, 50%, 45%, 40%, 35%, 30%, 25%, 20%, 15%, 10%, 5% or 2% of wild type activity. Activity, and changes in activity, may be measured by any techinique. In some embodiments, changes in activity is measured on a per cell mass basis.

In some embodiments, GCY1 is at least 80, 85, 90, 95, 99, or 100% identical to SEQ ID NO:1.

In some embodiments, the GCY1 gene comprises a nucleotide sequence that encodes an amino acid sequence at least 80, 85, 90, 95. 99, or 100% identical to the GCY1 amino acid sequence encoded by nucleotides 1-939 of SEQ ID NO: 1. In some embodiments, the GCY1 gene comprises a nucleotide sequence that encodes an amino acid sequence at least 80, 85, 90. 95, 99, or 100% identical to the GCY1 amino acid sequence of SEQ ID NO:2.

In some embodiments, the GCY1 gene product comprises an amino acid sequence at least 80, 85, 90, 95, 99, or 100% identical to the Gcy l amino acid sequence of residues 1-312 of SEQ ID NO:2.

In some embodiments, the Gcy1 protein comprises an amino acid sequence at least 80, 85, 90, 95, 99, or 100% identical to residues 1-312 of SEQ ID NO:2.

In some embodiments, Gcy1p is at least 80, 85, 90, 95, 99, or 100% identical to SEQ ID NO:2.

In some embodiments, the GCY1 gene comprises one or more mutations. In some embodiments, the GCY1 gene product comprises one or more mutations. In some embodiments, the Gy1p comprises one or more mutations. Said mutations may be additions, deletions, or substituted amino acid residue(s), or a combination thereof. In one aspect, provided herein are host cells that comprise modification or deletion of Gcy1p. In some embodiments, the host cells comprise the Gcy1p modification A35T.

In further embodiments, the host cells further comprise one or more deletions and/or one or more expressed genes useful for the production of vanillin and/or glucovanillin or for the reduction of the unwanted byproducts vanillyl alcohol and glucovanillyl alcohol.

In certain embodiments, the host cells further comprise deletion of HFD1. As described in the examples below, HFDI encodes the enzyme Hfd1 which is capable of converting vanillin to vanillic acid. Since vanillic acid is potentially toxic to cell strains, and an undesired impurity in the final product, it is an undesired fermentation side product. Further, accumulation of vanillic acid can make purification more difficult. In addition, the reverse reaction of vanillin to vanillic acid can introduce a futile cycle between vanillic acid and vanillin. Each forward reaction of vanillic acid to vanillin costs valuable cellular ATP and NADPH, which would then be wasted by the subsequent conversion of vanillin back to vanillic acid. In certain embodiments. the cell strains are S. cerevisiae. As described in the examples below, Hfd1 is the primary known enzyme responsible for converting vanillin to vanillic acid in S. cerevisiae. In cell strains other than S. cerevisiae, a homolog of HFD1 is deleted. Preferably, all copies of HFD1 are deleted. For instance, in haploid cells with one copy of HFD1, that copy is deleted. In diploid cells with two copies of HFD1, both copies are deleted. In any cells with multiple copies of HFD1. each copy is preferably deleted. The HFD1 gene(s) can be deleted by any technique apparent to those of skill in the art. Useful techniques include those based on homologous recombination and polymerase chain reaction (PCR).

In some embodiments, the host cells further comprise deletion of EXG1. EXG1 encodes encodes an endogenous yeast exoglucosidase 1, which efficiently hydrolyses vanillin glucoside.

In certain embodiments, the host cells further comprise deletion of ADH6. In host cells other than S. cerevisiae, a homolog of ADH6 is deleted. Preferably, all copies of ADH6 are deleted. For instance, in haploid cells with one copy of ADH6, that copy is deleted. In diploid cells with two copies of ADH6, both copies are deleted. In any cells with multiple copies of ADH6, each copy is preferably deleted. The ADH6 gene(s) can be deleted by any technique apparent to those of skill in the art. Useful techniques include those based on homologous recombination and polymerase chain reaction (PCR).

In certain embodiments, the host cells further comprise deletion of GRE2. In host cells other than S. cerevisiae, a homolog of GRE2 is deleted. Preferably, all copies of GRE2 are deleted. For instance, in haploid cells with one copy of GRE2, that copy is deleted. In diploid cells with two copies of GRE2, both copies are deleted. In any cells with multiple copies of GRE2, each copy is preferably deleted. The GRE2 gene(s) can be deleted by any technique apparent to those of skill in the art. Useful techniques include those based on homologous recombination and polymerase chain reaction (PCR).

In certain embodiments, the host cells furthert comprise deletion of SKY1. In host cells other than S. cerevisiae, a homolog of SKY1 is deleted. Preferably, all copies of SKY1 are deleted. For instance, in haploid cells with one copy of SKY1, that copy is deleted. In diploid cells with two copies of SKY1, both copies are deleted. In any cells with multiple copies of SKY1, each copy is preferably deleted. The SKY1 gene(s) can be deleted by any technique apparent to those of skill in the art. Useful techniques include those based on homologous recombination and polymerase chain reaction (PCR).

In certain embodiments, the host cells further comprise deletion of YGL039W. In host cells other than S. cerevisiae, a homolog of YGL039W is deleted. Preferably. all copies of YGL039W are deleted. For instance, in haploid cells with one copy of YGL039W, that copy is deleted. In diploid cells with two copies of YGL039W. both copies are deleted. In any cells with multiple copies of YGL039W, each copy is preferably deleted. The YGL039W gene(s) can be deleted by any technique apparent to those of skill in the art. Useful techniques include those based on homologous recombination and polymerase chain reaction (PCR).

In particular embodiments, the host cells further comprise enzymes of a pathway useful for the production of vanillin or glucovanillin. Such pathway enzymes have been described previously, including those described in Hansen et al., Appl. Environ. Microbiol. (2009) 75(9):2765-2774: U.S. Pat. Nos. 6,372,461 B1; 10,066,252 B1; 10,208,293 B2; each of which are incorporated by reference in their entireties.

In certain embodiments, the host cells further comprise a 3-dehydroquinate synthase, or AroB. Useful AroB genes and enzymes are known. Useful AroB polypeptides are also known. Useful AroB genes and enzymes include those of E. coli. Examples can be found at UniProtKB P07639. In preferred embodiments, the host cells further express or overexpress E. coli AroB.

In certain embodiments, the host cells further comprise a 3-dehydroquinate dehydratase, or AroD. Useful AroD genes and enzymes are known. Useful AroD polypeptides are also known. Useful AroD genes and enzymes include those of E. coli. Examples can be found at UniProtKB P05194. In preferred embodiments, the host cells further express or overexpress E. coli AroD.

In certain embodiments, the host cells further comprise a phospho-2-dehydro-3-deoxyheptonate aldolase, Tyr-sensitive, or AroF. Useful AroF genes and enzymes are known. Useful AroB polypeptides are also known. Useful AroF genes and enzymes include those of E. coli. Examples can be found at UniProtKB P00888. In preferred embodiments, the host cells further express or overexpress E. coli AroF. In certain embodiments, the AroF is feedback resistant (J. Bacteriol. November 1990 172:6581-6584, incorporated by reference in its entirety).

In certain embodiments, the host cells further comprise a 3-dehydroshikimate dehydratase, or AroZ. Useful AroZ genes and enzymes are known. Useful 3DSD polypeptides are also known. Useful AroZ genes and enzymes include those of Podospora pauciseta, Ustilago maydis, Rhodoicoccus jostii, Acinetobacter sp., Aspergillus niger and Neurospora crassa. Examples can be found at GenBank Accession Nos. CAD60599, XP_001905369.1, XP_761560.1, ABG93191.1, AAC37159.1, and XM_001392464. In preferred embodiments, the host cells further express or overexpress Podospora pauciseta AroZ.

In certain embodiments, the cell strains further comprise an ACAR. Useful ACAR genes and enzymes are known. Useful ACAR polypeptides are also known. In certain embodiments, the cell strains express one or more ACAR enzymes from one or more of the following organism sources: Actinokineospora spheciospongiae, Aspergillus terreus, Coccomyxa subellipsoidea, Gordonia effusa, Hypocrea jecorina. Kibdelosporangium sp. MJ126-NF4, Lichtheimia corymbifera, Metarhizium brunneum, Mycobacterium abscessus, Mycobacterium avium, Mycobacterium cosmeticum, Mycobacterium lepromatosis, Mycobacterium nebraskense, Mycobacterium obuense, Mycobacterium sp. MOTT36Y, Mycobacterium sp. URHB0044, Mycobacterium vaccae, Mycobacterium xenopi, Neurospora crassa, Nocardia brasiliensis, Nocardia gamkensis, Nocardia iowensis, Nocardia otitidiscaviarum, Nocardia seriolae, Nocardia terpenica, Nocardia vulneris, Purpureocillium lilacinum, Rhodococcus sp. Leaf258, Streptomyces sp. NRRL S-31, Talaromyces marneffei.

In certain embodiments, the host cells further comprise an PPTASE. Useful PPTASE genes and enzymes are known. Useful PPTASE polypeptides are also known. Useful PPTASE genes and enzymes include those of E. coli, Corynebacterium glutamicum, and Nocardia farcinica. Examples can be found at GenBank Accession Nos. NP_601186, BAA35224, and YP_120266. In preferred embodiments, the host cells further express or overexpress Cornybacterium glutamicum PPTASE.

In another aspect, provided herein are cell strains that express one or more heterologous O-methyltransferases (OMTs). As shown in FIG. 2, OMT catalyzes the conversion of protocatechuic acid (PCA) to vanillic acid and the conversin of PC aldehyde to vanillin. The OMT can be any OMT deemed useful by those of skill. In advantageous embodiments, the OMT has specificity for the correct —OH group of protocatechuic acid. In other words, in advantageous embodiments, the OMT forms more vanillic acid and less side product in this reactio n. As described herein, these OMTs provide excellent specificity for the correct —OH group and minimize formation of side product. In certain embodiments, the cell strains express one or more OMTs selected from the group consisting of OMTs from the following organism sources: Brachypodium distachyon. Brassica napus, Chelonia mydas, Cicer arietinum, Ciona intestinalis, Coccidioides posadasii, Cucumis sativus, Danio rerio, Dicentrarchus labrax, Esox lucius, Hordeum vulgare, Ictalurus punctatus, Medicago truncatula, Oryzias latipes, Osmerus mordax, Phoenix dactylifera, Setaria italica, Solanum tuberosum, Sorghum bicolor, Streptomyces sp. Root431. and Tuber melanosporum.

In certain embodiments, the host cells further comprise EAO. Useful EAO enzymes are known. Useful EAO genes and enzymes include those from Rhodococcus jostii. In certain embodiments. the EAO sequence is according to UniProtKB QOSBK1. In preferred embodiments, the host cells further express or overexpress Rhodococcus jostii EAO.

In certain embodiments, the cell strains are capable of glucosylating vanillin to form glucovanillin. Glucovanillin is a storage form of vanillin found in the vanilla pod. It is non-toxic to most organisms, including yeast, and has a higher solubility in water, as compared to vanillin. In addition, the formation of vanillin-β-D-glucoside most likely directs biosynthesis toward vanillin production. Useful UGT genes and enzymes for this conversion are known. Useful UGT enzymes according to the invention are classified under EC 2.4.1. Suitable UGT polypeptides include the UGT71C2, UGT72B1, UGT72E2, UGT84A2, UGT89B1, UGT85B1, and arbutin synthase polypeptides, at, for example. GenBank Accession Nos. AC0005496, NM_116337, and NM_126067. In certain embodiments. the cell strains further express or overexpress one or more of UGT71C2, UGT72B1, UGT72E2. UGT84A2, UGT89B1, UGT85B1, and arbutin synthase. In preferred embodiments, the cell strains further express or overexpress A. thaliana UGT72E2.

Overexpression can be according to any technique apparent to those of skill in the art. In certain embodiments, the genes are overexpressed from a promoter useful in the host cell. In certain embodiments, the genes are overexpressed from a S. cerevisiae promoter. In certain embodiments. the promoter is selected from the group consisting of pPGK1, pTDH3, pENO2, pADH1, pTPI1, pTEF1, pTEF2, pTEF3, pGAL1, pGAL2, pGAL7, pGAL10, GAL1, pRPL3, pRPL15A, pRPL4, pRPL8B, pSSA1, pSSB1, pCUP1, pTPS1, pHXT7, pADH2, pCYC1, and pPDA1. In certain embodiments. the genes are overexpressed from a GAL promoter. In certain embodiments. the genes are overexpressed from a promoter selected from the group consisting of pGAL1, pGAL2, pGAL7, pGAL10, and variants thereof.

In certain embodiments, one, some, or all of the heterologous promoters in the host cells are inducible. The inducible promoter system can be any recognized by those of skill in the art. In particular embodiments. the promoters are inducible by maltose. In an advantageous embodiment. the host cells comprise a GAL regulon that is inducible by maltose. Examples of the Gal regulon which are further repressed or induced by maltose are described in PCT Application Publications WO2015/020649. WO2016/210343, and WO2016210350. each of which is incorporated by reference in its entirety.

In certain embodiments. a maltose switchable strain is built on top of a non-switchable strain by chromosomally integrating a copy of GAL80 under the control of a maltose-responsive (MAL) promoter such as pG2MAL and pMAL32, and pMAL1, pMAL2, pMAL11, pMAL12, pMAL31, and pGMAL. Specific examples of pG2MAL include: pG2MAL_v1 (SEQ ID NO:3), pG2MAL_v2 (SEQ ID NO:4), pG2MAL_v3 (SEQ ID NO:5), pG2MAL_v5 (SEQ ID NO:6), pG2MAL_v16 (SEQ ID NO:7), pG2MAL_v7 (SEQ ID NO:8), pG2MAL_v8 (SEQ ID NO:9), pG2MAL_v19 (SEQ ID NO:10), and pG2MAL_v10 (SEQ ID NO:11). Specific examples of pMAL32 include pMAL32 (SEQ ID NO: 12) and pMAL32_v1 (SEQ ID NO: 13). Specific examples of pMAL1, pMAL2, pMAL11, pMAL12, pMAL31, and pGMAL include pMAL1 (SEQ ID NO: 14), pMAL2 (SEQ ID NO:15), pMAL11 (SEQ ID NO: 16), pMAL12 (SEQ ID NO:17), pMAL31 (SEQ ID NO:18), pGMAL_v5 (SEQ ID NO:19), pGMAL_v6 (SEQ ID NO:20), pGMAL_v7 (SEQ ID NO:21), pGMAL_v9 (SEQ ID NO:22), pGMAL_v10 (SEQ ID NO:23), pGMAL_v1I (SEQ ID NO:24), pGMAL_v12 (SEQ ID NO:25). pGMAL_v13 (SEQ ID NO:26), pGMAL_v14 (SEQ ID NO:27), pGMAL_v15 (SEQ ID NO:28), pGMAL_v16 (SEQ ID NO:29), pGMAL_v17 (SEQ ID NO:30). pGMAL_v18 (SEQ ID NO:31).

In certain embodiments, the GAL80 gene product is mutated for temperature sensitivity, e.g. to facilitate further control. In certain embodiments, the GAL80 gene product is fused to a temperature-sensitive polypeptide. In certain embodiments. the GAL80 gene product is fused to a temperature-sensitive DHFR polypeptide or fragment. Additional description of switchable farnesene producing switchable strains are described in U.S. Patent Application Publication No. US 2016/0177341 and PCT Application Publication No. WO 2016/210350, each of which is incorporated herein by reference in its entirety.

For each of the polypeptides and nucleic acids described above, the host cells can comprise variants thereof. In certain embodiments, the variant can comprise up to 15, 10, 9, 8, 7, 6, 5, 4, 3, 2, or 1 amino acid substitutions relative to the relevant polypeptide. In certain embodiments, the variant can comprise up to 15, 10, 9, 8, 7, 6, 5, 4, 3, 2, or 1 conservative amino acid substitutions relative to the reference polypeptide. In certain embodiments, any of the nucleic acids described herein can be optimized for the host cell, for instance codon optimized. Variants and optimization are described in detail below.

In certain embodiments, the additional enzymes are native, unless specified otherwise above. Native enzymes can be expressed from codon optimized nucleic acids. In advantageous embodiments, the additional enzymes are heterologous. In certain embodiments, two or more enzymes can be combined in one polypeptide.

### Cell Strains

Host cells useful compositions and methods provided herein include archae, prokaryotic, or eukaryotic cells.

Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia coli cell.

Suitable archae hosts include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella. Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis. Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the host microbe is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the host microbe is Saccharomyces cerevisiae. In some embodiments, the host is a strain of Saccharomyces cerevisiae selected from the group consisting of Baker's yeast, CEN.PK, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the host microbe is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the host microbe is a microbe that is suitable for industrial fermentation. In particular embodiments, the microbe is conditioned to subsist under high solvent concentration, high temperature, high pressure, expanded substrate utilization. nutrient limitation, osmotic stress due to sugar and salts, acidity, sulfite and bacterial contamination, or combinations thereof, which are recognized stress conditions of the industrial fermentation environment.

### Methods of Producing Vanillin or Glucovanillin

In another aspect, provided herein is a method for the production of a vanillin or glucovanillin, the method comprising the steps of: (a) culturing a population of any of the genetically modified host cells described herein that are capable of producing a vanillin or glucovanillin in a medium with a carbon source under conditions suitable for making the vanillin or glucovanillin compound: and (b) recovering said vanillin or glucovanillin compound from the medium. Those of skill will recognize that the amount of a compound produced can be evaluated by measuring the amount of the compound itself, or more preferably the amount of the compound and derivatives of the compound. For instance, the amount of vanillin produced can be evaluated from the total amount of vanillin, vanillyl alcohol, glucovanillin, and glucovanillyl alcohol produced.

In some embodiments, the genetically modified host cell produces an increased amount of the vanillin or glucovanillin, or derivative thereof such as vanillyl alcohol or glucovanillyl alcohol, compared to a parent cell not comprising the one or more modifications, or a parent cell comprising only a subset of the one or more modifications of the genetically modified host cell, but is otherwise genetically identical. In some embodiments, the increased amount is at least 1%, 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, production, and/or productivity, in grams per liter of cell culture, milligrams per gram of dry cell weight, on a per unit volume of cell culture basis, on a per unit dry cell weight basis, on a per unit volume of cell culture per unit time basis, or on a per unit dry cell weight per unit time basis.

In some embodiments, the host cell produces an elevated level of a vanillin or glucovanillin, or derivative thereof such as vanillyl alcohol or glucovanillyl alcohol, that is greater than about 0.25 grams per liter of fermentation medium. In some embodiments, the host cell produces an elevated level of a vanillin or glucovanillin, or derivative thereof such as vanillyl alcohol or glucovanillyl alcohol, that is greater than about 0.5 grams per liter of fermentation medium. In some embodiments, the host cell produces an elevated level of a vanillin or glucovanillin, or derivative thereof such as vanillyl alcohol or glucovanillyl alcohol, that is greater than about 0.75 grams per liter of fermentation medium. In some embodiments, the host cell produces an elevated level of a vanillin or glucovanillin, or derivative thereof such as vanillyl alcohol or glucovanillyl alcohol, that is greater than about 1 grams per liter of fermentation medium. In some embodiments, the host cell produces an elevated level of a vanillin or glucovanillin, or derivative thereof such as vanillyl alcohol or glucovanillyl alcohol, that is greater than about 5 grams per liter of fermentation medium. In some embodiments, the host cell produces an elevated level of a vanillin or glucovanillin, or derivative thereof such as vanillyl alcohol or glucovanillyl alcohol, that is greater than about 10 grams per liter of fermentation medium. In some embodiments, the vanillin or glucovanillin, or derivative thereof such as vanillyl alcohol or glucovanillyl alcohol, is produced in an amount from about 10 to about 50 grams, from about 10 to about 15 grams, more than about 15 grams, more than about 20 grams, more than about 25 grams, or more than about 30 grams per liter of cell culture.

In some embodiments, the host cell produces an elevated level of a vanillin or glucovanillin, or derivative thereof such as vanillyl alcohol or glucovanillyl alcohol, that is greater than about 50 milligrams per gram of dry cell weight. In some such embodiments, the vanillin or glucovanillin, or derivative thereof such as vanillyl alcohol or glucovanillyl alcohol, is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the host cell produces an elevated level of a vanillin or glucovanillin, or derivative thereof such as vanillyl alcohol or glucovanillyl alcohol. that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of vanillin or glucovanillin, or derivative thereof such as vanillyl alcohol or glucovanillyl alcohol, produced by a parent cell, on a per unit volume of cell culture basis.

In some embodiments, the host cell produces an elevated level of a vanillin or glucovanillin, or derivative thereof such as vanillyl alcohol or glucovanillyl alcohol, that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of vanillin or glucovanillin, or derivative thereof such as vanillyl alcohol or glucovanillyl alcohol, produced by the parent cell, on a per unit dry cell weight basis.

In some embodiments, the host cell produces an elevated level of a vanillin or glucovanillin, or derivative thereof such as vanillyl alcohol or glucovanillyl alcohol, that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of vanillin or glucovanillin, or derivative thereof such as vanillyl alcohol or glucovanillyl alcohol, produced by the parent cell. on a per unit volume of cell culture per unit time basis.

In some embodiments, the host cell produces an elevated level of a vanillin or glucovanillin, or derivative thereof such as vanillyl alcohol or glucovanillyl alcohol, that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of vanillin or glucovanillin, or derivative thereof such as vanillyl alcohol or glucovanillyl alcohol, produced by the parent cell. on a per unit dry cell weight per unit time basis.

In most embodiments, the production of the elevated level of vanillin or glucovanillin by the host cell is inducible by the presence of an inducing compound or the absence of a repressing compound. Such a host cell can be manipulated with ease in the absence of the inducing compound or the presence of the repressing compound. The inducing compound is then added, or the repressing compound is diminished, to induce the production of the elevated level of vanillin or glucovanillin by the host cell. In other embodiments, production of the elevated level of vanillin or glucovanillin by the host cell is inducible by changing culture conditions, such as, for example, the growth temperature, media constituents. and the like. In certain embodiments, the vanillin-producing enzymes are repressed by maltose during a growth phase of the cells, and the vanillin-producing enzymes are expressed during an expression phase of the fermentation. Useful promoters and techniques are described in US 2018/0171341 A1, incorporated by reference in its entirety.

In certain embodiments, provided herein is vanillin or glucovanillin, or both, produced by the methods herein. In certain embodiments, provided herein is vanillin having a unique isotope profile, compared to standard. In certain embodiments, provided herein is vanillin having a unique carbon isotope profile, compared to standard. Carbon isotope profiles are measured according to standard techniques, for instance those described in the examples herein. The standard can be any standard deemed suitable by those of skill. In certain embodiments, the standard is oxalic acid for measurement of ¹⁴C activities. In certain embodiments, the ¹⁴C activities are reported as disintegrations per min per gram of carbon (dpm/g of C) which can be used to differentiate between petroleum derived vanillin (dpm/g of C approaching 0) versus derived from a plant source which typically gives 15-16 dpm/g of C. In certain embodiments, provided herein is vanillin having a ¹⁴C activity of about 12.9 to about 14.1 dpm/g. In certain embodiments, provided herein is vanillin having a ¹⁴C activity of about 12.9 dpm/g. In certain embodiments, provided herein is vanillin having a ¹⁴C activity of about 14.1 dpm/g. In certain embodiments, carbon isotope ratios are expressed as ‰=[(Rₛₐₘₚₗₑ/R_{standard})−1]×10³, where R=¹³C/¹²C is expressed relative to the Pee Dee Belemnite (PDB) standard. In certain embodiments, provided herein is vanillin having a bulk δ¹³C deviation from PDB standard of about −14.8 to about −12.8 permil (‰). In certain embodiments, provided herein is vanillin having a bulk δ¹³C deviation from PDB standard of about −12.8 permil (‰). In certain embodiments, provided herein is vanillin having a bulk δ¹³C deviation from PDB standard of about −14.8 permil (‰). Hydrogen isotope profiles are measured according to standard techniques, for instance those described in the examples herein. The hydrogen isotope standard can be any standard deemed suitable by those of skill. In certain embodiments. provided herein the standard is Standard Mean Ocean Water (SMOW). In certain embodiments, hydrogen isotope ratios are expressed as ‰=[(Rₛₐₘₚₗₑ/R_{standard})−1]×10³, where R=²H/¹H is expressed relative to the Standard Mean Ocean Water (SMOW) standard. In certain embodiments, provided herein is vanillin having a bulk 82H deviation from SMOW standard of about −150 to about −124 permil (‰). In certain embodiments, provided herein is vanillin having a bulk δ²H deviation from SMOW standard of about −150 permil (‰). In certain embodiments, provided herein is vanillin having a bulk δ²H deviation from SMOW standard of about −124 permil (‰).

### Culture Media and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature. and requirements for aerobic, microaerobic. or anaerobic conditions, depending on the specific requirements of the host cell, the fermentation, and the process.

The methods of producing vanillin and/or glucovanillin provided herein may be performed in a suitable culture medium in a suitable container, including but not limited to a cell culture plate, a microtiter plate, a flask. or a fermentor. Further, the methods can be performed at any scale of fermentation known in the art to support industrial production of microbial products. Any suitable fermentor may be used including a stirred tank fermentor. an airlift fermentor, a bubble fermentor, or any combination thereof. In particular embodiments utilizing Saccharomyces cerevisiae as the host cell, strains can be grown in a fermentor as described in detail by Kosaric, et al. in Ullmann's Encyclopedia of Industrial Chemistry, Sixth Edition, Volume 12, pages 398-473, Wiley-VCH Verlag Gmbh & Co. KDaA, Weinheim, Germany.

In some embodiments, the culture medium is any culture medium in which a genetically modified microorganism capable of producing vanillin or glucovanillin can subsist, i.e., maintain growth and viability. In some embodiments, the culture medium is an aqueous medium comprising assimilable carbon, nitrogen and phosphate sources. Such a medium can also include appropriate salts, minerals, metals and other nutrients. In some embodiments, the carbon source and some or all of the essential cell nutrients are added incrementally or continuously to the fermentation media. In certain embodiments, a subset of the essential nutrients are maintained in excess while a few, e.g. one or two, required nutrients are maintained at about the minimum levels needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. In some embodiments, the suitable medium is supplemented with one or more additional agents, such as. for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose. trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate, ethanol, and glycerol.

The concentration of a carbon source, such as glucose, in the culture medium is sufficient to promote cell growth, but is not so high as to repress growth of the microorganism used. Typically, cultures are run with a carbon source, such as glucose, being added at levels to achieve the desired level of growth and biomass. In other embodiments, the concentration of a carbon source, such as glucose, in the culture medium is greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result. the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

The culture medium can also contain a suitable sulfur source. Preferred sulfur sources include, but are not limited to, sulfate salts such as ammonium sulfate ((NH₄)₂SO₄), magnesium sulfate (MgSO₄), potassium sulfate (K₂SO₄), and sodium sulfate (Na₂SO₄) and mixtures thereof. Typically, the concentration of sulfate in the culture medium is greater than about 1.0 g/L, preferably greater than about 3.0 g/L and more preferably greater than about 10.0 g/L. Beyond certain concentrations, however, the addition of sulfate to the culture medium is not advantageous for the growth of the microorganisms. Accordingly, the concentration of sulfate in the culture medium is typically less than about 50 g/L, preferably less than about 30 g/L and more preferably less than about 20 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations. however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments. the fermentation is carried out in fed-batch mode. In such a case, some of the components of the medium are depleted during culture during the production stage of the fermentation. In some embodiments, the culture may be supplemented with relatively high concentrations of such components at the outset, for example, of the production stage, so that growth and/or vanillin or glucovanillin production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of vanillin or glucovanillin. For example, prior to inoculation of the culture medium with an inoculum, the culture medium can be brought to and maintained at a temperature in the range of from about 20° ° C. to about 45° C., preferably to a temperature in the range of from about 25° C. to about 40° C. In certain embodiments, the cells are eukaryotic, e.g. yeast, and the temperature is in the range of from about 28° C. to about 34° C. In certain embodiments, the cells are prokaryotic, e.g. bacteria, and the temperature is in the range of from about 35° ° C. to about 40° C., for instance 37° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0. In certain embodiments, the cells are eukaryotic, e.g. yeast, and the pH is preferably from about 4.0 to about 6.5. In certain embodiments, the cells are prokaryotic, e.g. bacteria, and the pH is from about 6.5 to about 7.5, e.g. about 7.0.

In some embodiments, the carbon source concentration, such as the glucose, fructose or sucrose, concentration, of the culture medium is monitored during culture. Carbon source concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. The carbon source concentration is typically maintained below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose, fructose, or sucrose is used as a carbon source the glucose, fructose, or sucrose is preferably fed to the fermentor and maintained below detection limits. Alternatively. the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a carbon source solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

Other suitable fermentation medium and methods are described in, e.g., WO 2016/196321.

### Fermentation Compositions

In another aspect, provided herein are fermentation compositions comprising a genetically modified host cell described herein and vanillin and/or glucovanillin produced from the genetically modified host cell. The fermentation compositions may further comprise a medium. In certain embodiments, the fermentation compositions comprise a genetically modified host cell, and further comprise vanillin or glucovanillin. In certain embodiments, the fermentation compositions provided herein comprise vanillin as a major component of the vanillin and/or glucovanillin produced from the genetically modified host cell. In certain embodiments, the fermentation compositions provided herein comprise glucovanillin as a major component of the vanillin and/or glucovanillin produced from the genetically modified host cell.

### Recovery of Vanillin and/or glucovanillin

Once the vanillin or glucovanillin is produced by the host cell, it may be recovered or isolated for subsequent use using any suitable separation and purification methods known in the art. In some embodiments, a clarified aqueous phase comprising the vanillin or glucovanillin is separated from the fermentation by centrifugation or filtration. In certain embodiments, flocculants and coagulants are added to the clarified aqueous phase, for instance, to the clarified aqueous phase.

The vanillin or glucovanillin produced in these cells may be present in the culture supernatant and/or associated with the host cells. In embodiments where some of the vanillin or glucovanillin is associated with the host cell, the recovery of the vanillin or glucovanillin may comprise a method of improving the release of the vanillin and/or glucovanillin from the cells. In some embodiments, this could take the form of washing the cells with hot water or buffer treatment, with or without a surfactant, and with or without added buffers or salts. In some embodiments, the temperature is any temperature deemed suitable for releasing the vanillin and/or glucovanillin. In some embodiments, the temperature is in a range from 40 to 95° C.: or from 60 to 90° C.: or from 75 to 85° C. In some embodiments, the temperature is 40, 45, 50, 55, 65, 70, 75, 80, 85, 90, or 95° C. In some embodiments physical or chemical cell disruption is used to enhance the release of vanillin and/or glucovanillin from the host cell. Alternatively and/or subsequently, the vanillin or glucovanillin in the culture medium can be recovered using an isolation unit operations including, but not limited to solvent extraction. membrane clarification, membrane concentration, adsorption, chromatography, evaporation. chemical derivatization, crystallization, and drying.

### Methods of Making Genetically Modified Cells

Also provided herein are methods for producing a host cell that is genetically engineered to comprise one or more of the modifications described above, e.g., one or more nucleic heterologous nucleic acids and/or biosynthetic pathway enzymes, e.g., for a vanillin or glucovanillin compound. Expression of a heterologous enzyme in a host cell can be accomplished by introducing into the host cells a nucleic acid comprising a nucleotide sequence encoding the enzyme under the control of regulatory elements that permit expression in the host cell. In some embodiments, the nucleic acid is an extrachromosomal plasmid. In other embodiments, the nucleic acid is a chromosomal integration vector that can integrate the nucleotide sequence into the chromosome of the host cell. In other embodiments, the nucleic acid is a linear piece of double stranded DNA that can integrate via homology the nucleotide sequence into the chromosome of the host cell.

Nucleic acids encoding these proteins can be introduced into the host cell by any method known to one of skill in the art without limitation (see, for example, Hinnen et al. (1978) Proc. Natl. Acad. Sci. USA 75:1292-3: Cregg et al. (1985) Mol. Cell. Biol. 5:3376-3385: Goeddel et al. eds, 1990. Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology. Greene Publishing Associates and Wiley Interscience, NY). Exemplary techniques include, but are not limited to, spheroplasting, electroporation, PEG 1000 mediated transformation, and lithium acetate or lithium chloride mediated transformation.

The amount of an enzyme in a host cell may be altered by modifying the transcription of the gene that encodes the enzyme. This can be achieved for example by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked. Alternatively or in addition, the copy number of an enzyme in a host cell may be altered by modifying the level of translation of an mRNA that encodes the enzyme. This can be achieved for example by modifying the stability of the mRNA. modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell can be altered in a number of ways, including, but not limited to, expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower Kcat or a lower or higher Km for the substrate, or expressing an altered form of the enzyme that is more or less affected by feed-back or feed-forward regulation by another molecule in the pathway.

In some embodiments, a nucleic acid used to genetically modify a host cell comprises one or more selectable markers useful for the selection of transformed host cells and for placing selective pressure on the host cell to maintain the foreign DNA.

In some embodiments, the selectable marker is an antibiotic resistance marker. Illustrative examples of antibiotic resistance markers include, but are not limited to, the BLA. NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NATI gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin: the SMR1 gene product confers resistance to sulfometuron methyl; the CAT gene product from Tn9 transposon confers resistance to chloramphenicol: the mouse dhfr gene product confers resistance to methotrexate: the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). In some embodiments, the antibiotic resistance marker is deleted after the genetically modified host cell disclosed herein is isolated.

In some embodiments, the selectable marker rescues an auxotrophy (e.g., a nutritional auxotrophy) in the genetically modified microorganism. In such embodiments, a parent microorganism comprises a functional disruption in one or more gene products that function in an amino acid or nucleotide biosynthetic pathway and that when non-functional renders a parent cell incapable of growing in media without supplementation with one or more nutrients. Such gene products include, but are not limited to, the HIS3, LEU2, LYS1, LYS2, MET15, TRP1, ADE2, and URA3 gene products in yeast. The auxotrophic phenotype can then be rescued by transforming the parent cell with an expression vector or chromosomal integration construct encoding a functional copy of the disrupted gene product, and the genetically modified host cell generated can be selected for based on the loss of the auxotrophic phenotype of the parent cell. Utilization of the URA3, TRP1, and LYS2 genes as selectable markers has a marked advantage because both positive and negative selections are possible. Positive selection is carried out by auxotrophic complementation of the URA3. TRP1, and LYS2 mutations, whereas negative selection is based on specific inhibitors, i.e .. 5-fluoro-orotic acid (FOA), 5-fluoroanthranilic acid, and aminoadipic acid (aAA), respectively, that prevent growth of the prototrophic strains but allows growth of the URA3, TRP1, and LYS2 mutants, respectively. In other embodiments, the selectable marker rescues other non-lethal deficiencies or phenotypes that can be identified by a known selection method.

Described herein are specific genes and proteins useful in the methods, compositions and organisms of the disclosure; however it will be recognized that absolute identity to such genes is not necessary. For example. changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp (www.idtdna.com/CodonOptfrom) from Integrated DNA Technologies.

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life. as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DVA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50%, 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%. even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L). Alanine (A), Valine (V). and 6) Phenylalanine (F), Tyrosine (Y), Tryptophan (W).

Sequence homology for polypeptides, which is also referred to as percent sequence identity, is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous UDP glycosyltransferases, or any biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation. design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

EXAMPLES

### Example 1. Yeast Transformation Methods

Each DNA construct is integrated into Saccharomyces cerevisiae (CEN.PK2) with standard molecular biology techniques in an optimized lithium acetate (LiAc) transformation. Briefly, cells are grown overnight in yeast extract peptone dextrose (YPD) media at 30° C. with shaking (200 rpm), diluted to an OD600 of 0.1 in 100 mL YPD, and grown to an OD600 of 0.6-0.8. For each transformation, 5 mL of culture is harvested by centrifugation, washed in 5 mL of sterile water, spun down again, resuspended in 1 mL of 100 mM LiAc, and transferred to a microcentrifuge tube. Cells are spun down (13,000× g) for 30 seconds, the supernatant is removed, and the cells are resuspended in a transformation mix consisting of 240 μL 50% PEG, 36 μL 1 M LiAc, 10 μL boiled salmon sperm DNA, and 74 μL of donor DNA. Following a heat shock at 42°C. for 40 minutes, cells are recovered overnight in YPD media before plating on selective media. DNA integration is confirmed by colony PCR with primers specific to the integrations.

### Example 2: Generation of a Strain with High Flux to Glucovanillin

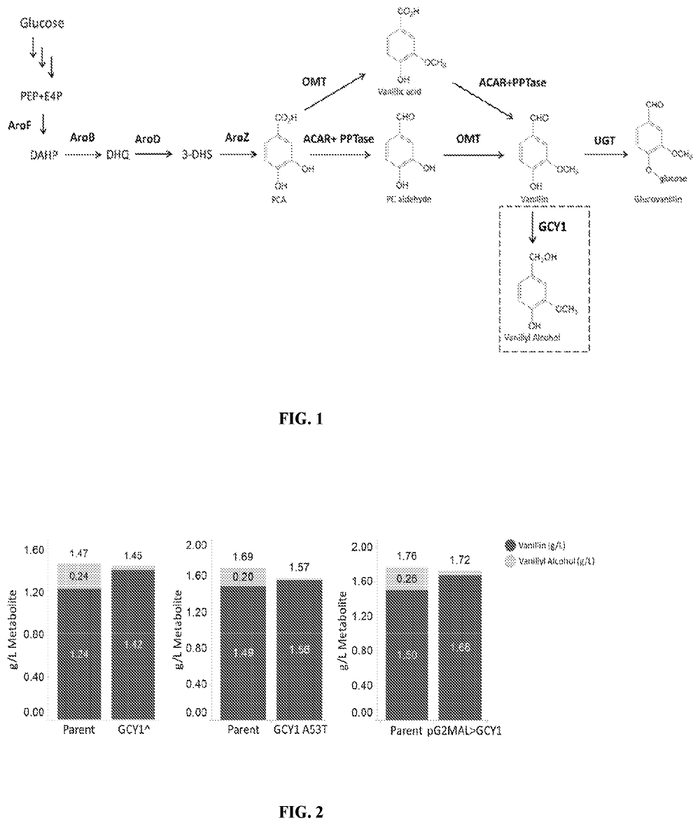

FIG. 1 shows an exemplary biosynthetic pathway to produce glucovanillin from central carbon metabolites erythrose-4-phosphate (E4P) and phosphoenylpyruvate (PEP). A glucovanillin production strain was created from a wild-type Saccharomyces cerevisiae strain (CEN.PK) by expressing heterologous genes from native GAL promoters. This strain comprised the following chromosomally integrated heterologous genes: AroF, AroB, AroD, AroZ, OMT, ACAR, PPTase, UGT, and EAO. The following subset of these genes include two chromosomally integrated copies: AroZ and UGT. The following subset of these genes include four chromosomally integrated copies: OMT.

In addition, native yeast genes were deleted in order to prevent degradation of vanillin and glucovanillin into unwanted byproducts including oxidation to vanillic acid (HFD1), and hydrolysis of glucovanillin to vanillin (EXG1).

In addition, the native yeast gene GCY1 (YOR120W) was altered in order to prevent reduction of vanillin and glucovanillin into the unwanted byproducts vanillyl alcohol and glucovanillyl alcohol. In one aspect, the entire ORF (open reading frame) of GCY1 (YOR120W) was deleted. In one aspect, the amino acid at position 35 (Alanine) was mutated to Threonine. In one aspect, the promoter of GCY1 (the 100 base pairs immediately upstream of the start codon (ATG)) are replaced with a MAL promoter (e.g., the pG2MAL promoter or the pMAL32 promoter) as described previously in U.S. Patent Application Publication No. US 20180186841 and WO 2016/210343, the entire contents of both of which are herein incorporated by reference.

### Example 3. Yeast Culturing Conditions in 96-Well Plates

Yeast colonies were picked into 96-well microtiter plates containing Bird Seed Media (BSM) (100 ml/L Bird Batch (Potassium phosphate 80 g/L, Ammonium Sulfate 150 g/L, Magnesium Sulfate 61.5 g/L) 5ml/L Trace Metal Solution (0.5M EDTA 160 mL/L, Zinc sulfate heptahydrate 11.5 g/L, Copper Sulfate 0.64 g/L. Manganese(II) chloride 0.64 g/L, Cobalt(II) Chloride Hexahydrate 0.94 g/L, Sodium molybdate 0.96 g/L, Iron(II) sulfate 5.6 g/L, Calcium Chloride dihydrate 5.8 g/L) 12 mL/L Birds Vitamins 2.0 (Biotin 0.05 g/L, p-Aminobenzoic Acid 0.2 g/L, D-Pantothenic Acid 1 g/L, Nicotinic Acid 1 g/L, Myoinositol 25 g/L, Thiamine HCl 1 g/L, Pyridoxine HCl 1 g/L, Succinic Acid 6 g/L, 1 g/L Lysine) with 1.9% Maltose and 0.1% Glucose. Cells were cultured at 30° C. in a high capacity microtiter plate incubator shaking at 1000 rpm and 80% humidity for 3 days until the cultures reached carbon exhaustion. The growth-saturated cultures were subcultured into fresh plates containing BSM with 4% sucrose and 1 g/L lysine by taking 14.4 μL from the saturated cultures and diluting into 360 μL of fresh media. Cells in the production media were cultured at 30° C. in a high capacity microtiter plate shaker at 1000 rpm and 80% humidity for additional 3 days prior to extraction and analysis. Biomass density was measured by optical density at 600 nm

### Example 4. Quantification of Vanillin and Vanillyl Alcohol

To quantify the amount of vanillin and vanillyl alcohol produced, the samples were first treated with a commercially available beta-glucosidase to convert glucovanillin into vanillin and gluco-vanillyl alcohol into vanillyl alcohol for analysis. Samples were then analyzed on a Agilent Vanquish™ Flex Binary UHPLC System with a diode array detector with the following program:
Mobile phase (A): 1.4% sulfuric acid v/v in water
Mobile phase (B): 100% acetonitrile

Gradient is as follows (gradient time, (min) mobile phase A, (%)): ((0.00, 88), (0.05, 88), (1.25, 85), (2.25, 83), (3.0, 82), (3.5, 88), (4.0, 88)). Flow rate was 1.

### Example 5. Fermentation Media and Conditions

Approximately five single colonies (1 mm loopful) of a yeast strain containing the desired genetic modifications was transferred to a 250 mL baffled flask containing 50 mL of YFS1255 BSM 3.5 (100 mL/L 1× Bird Batch (80 g/L KH₂PO₄, 150 g/L (NH₄)₂SO₄, 61.5 g/L MgSO₄*7H₂O), 3 mL/L Birds Vitamins 3.5, 4× (0.2 g/L biotin, 0.8 g/L para-Aminobenzoic Acid, 4 g/L nicotinic acid, 10 g/L myoinositol, 4 g/L pyridozine HCl, 4 g/L thiamine HCl, 4 g/L calcium pantothenate), 5 mL/L Bird TM 2× (11.5 g/L ZnSO4*7H₂O, 0.64 g/L CuSO₄, 0.64 MnCl₂*4H₂O, 0.94 g/L CoCl₂*6H₂O, 0.96 g/L Na₂MoO₄*2H₂O, 5.6 g/L FeSO₄*7H₂O, 5.8 CaCl₂*2H₂O, 160m/L 0.5M EDTA), 5g/L lysine, 40 g/L maltose, 20 g/L sucrose, 100 mL/L 0.5M succinate buffer). The cells were grown in a shaker at 28° C., 200 RPM for 21 hours (SF0 culture).

The OD of SF0 is then measured and used to calculate an inoculation volume for the next shake flask, SF1. The inoculation volume is based on the doubling time of 2.5 hours and the target OD of 3. SF1 is a 1000 mL baffled flask containing 250 mL of YFS1255 BSM 3.5. SF1 was incubated in a shaker at 28° C., 200 RPM for 23 hours.

The OD of SF1 is then measured and used to calculate an inoculation volume for the next shake flask, SF2. The inoculation volume is based on the doubling time of 2.5 hours and the target OD of 3. SF2 is a 1000 mL baffled flask containing 250 mL of YFS1255 BSM 3.5. SF2 was incubated in a shaker at 28° C., 200 RPM for 23 hours.

The OD of SF2 is then measured and used to calculate an inoculation volume for the next shake flask, SF3. The inoculation volume is based on the doubling time of 2.5 hours and the target OD of 7. SF3 is a 2000mL baffled flask containing 250 mL of YFS1255 BSM 3.5. SF3 was incubated n in a shaker at 28° C., 200 RPM for 24 hours.

If after 24 hours growth the OD of SF3 is less than 3, the flask is returned to the shaker until the OD is greater than 3. If after 24 hours growth the OD of SF3 is greater than 3, then 60 mL are transferred into a 0.5 L manufacturing fermentor (the MFA) containing 240 mL of YFS1255 BSM 3.5 MF media (30 ml/L 10× Bird Batch YMC0228 (80 g/L KH₂PO₄, 70 g/L (NH₄)₂SO₄, 61.5 g/L MgSO₄*7H₂O), 1.6 ml/L 4× Bird Vitamins 3.5 with 5× PABA YMC0237 (0.2 g/L biotin, 4 g/L para-Aminobenzoic Acid, 4 g/L nicotinic acid, 10 g/L myoinositol. 4 g/L pyridozine HCl, 4 g/L thiamine HCI, 4 g/L calcium pantothenate), 3 ml/L 2× Bird™ YMC0079 (11.5 g/L ZnSO₄*7H₂O, 0.64 g/L CuSO₄, 0.64 MnCl₂*4H₂O, 0.94 g/L CoCl₂*6H₂O, 0.96 g/L Na₂MoO₄*2H₂O, 5.6 g/L FeSO₄*7H₂O, 5.8 CaCl₂*2H2O, 160 m/L 0.5M EDTA), 0.72 g/L Lysine and 1.8 g/L Maltose).

The carbon feed to the fermentor is comprised of sucrose. An initial pulse of 10 g TRS/L (total reducing sugars per liter) sugar was delivered at 8.5 g/L/h. The feed rate is changed to 1 g/L/h once the 10 g TRS/L were delivered to the tank, and increased to 8.5 g/L/h when the culture runs out of carbon indicated by a DO value rise. The sugar feed rate is continuously adjusted to satisfy the culture demand of carbon. The fermentation was run microaerobically at a constant temperature of 30° C. and constant pH of 5.0 (controlled by ammonium hydroxide additions). The agitation is controlled to maintain an oxygen utilization rate of 110 mmol O₂/L/h for the remainder of the fermentation. Culture was removed daily for sampling and to prevent overflow: Salts, trace metals, and vitamins were also added daily. 0.1 mL Pluronic® L61 antifoam was added to the fermentation media at the beginning and subsequently added as needed. The amount of glucovanillin produced and the total sugar consumed by the cells was monitored daily and the ratio of these two values (i.e., the product yield off of sugar) was determined for each 24 hour period. The fermentor was run for 7 days.

### Example 6. Altering the Expression of the Native GCY1 Protein Increases the Amount of Total Vanillin and Decreases the Amount of Gluco-Vanillyl Alcohol and Vanillyl Alcohol Produced

When the native yeast gene GCY1 (YOR120W) was altered in strains producing vanillin and glucovanillin there was a decrease in the amount of the undesired byproducts vanillyl alcohol and gluco-vanillyl alcohol. In addition, there was an increase in the amount of glucovanillin and vanillin (total vanillin) produced. Because all samples are first treated with a commercially available beta-glucosidase before measurement, only vanillin and vanillyl alcohol are directly measured. In all aspects, the amount of vanillyl alcohol produced by the GCY1\hat{ } strains in 96-well plates decreases to quantities that are below the linear range for the assay to measure vanillyl alcohol (less than 0.12 g/L).

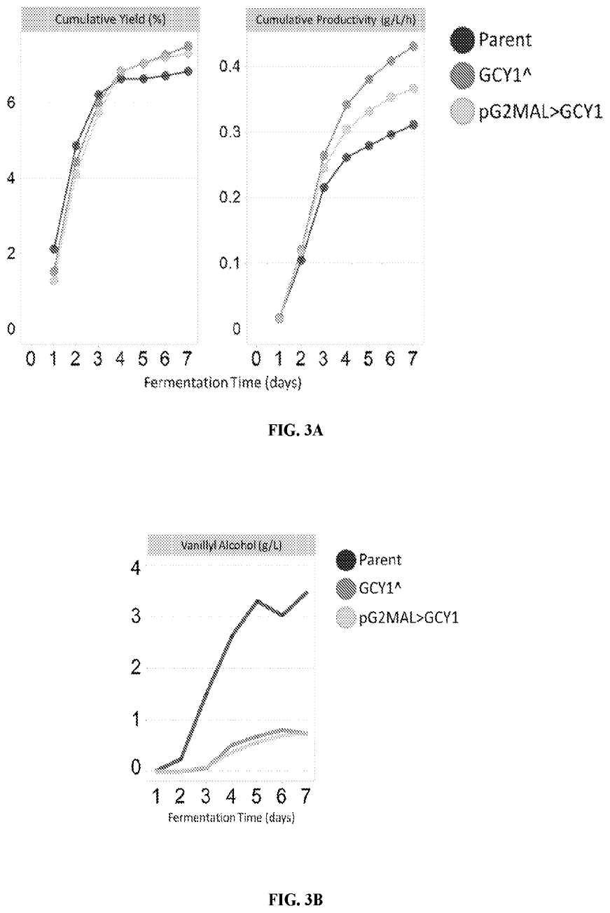

In one aspect, the entire ORF (open reading frame) of GCY1 (YOR120W) was deleted (FIG. 2 left) and the amount of vanillyl alcohol produced by the mutant strain decreased from 0.24 g/L to almost none while the amount of vanillin produced increased from 1.24 g/L to 1.42 g/L. In one aspect, the amino acid at position 35 (Alanine) was mutated to Threonine (FIG. 2 middle) and the amount of vanillyl alcohol produced by the mutant strain decreased from 0.20 g/L to almost none while the amount of vanillin produced increased from 1.49 g/L to 1.56 g/L. In one aspect, the promoter of GCY1 (the 100 base pairs immediately upstream of the start codon (ATG)) are replaced with the pG2MAL promoter (FIG. 3 right) described previously in U.S. Patent Application Publication No. US20180186841 and the amount of vanillyl alcohol produced by the mutant strain decreased from 0.26g/L to almost none while the amount of vanillin produced increased from 1.5 g/L to 1.68 g/L.

Vanillyl alcohol titers in fermentation are typically significantly higher than titers in a 96-well plate batch culture. Therefore, using fermentation we can more accurately quantify how much the alteration of the GCYI gene affects the vanillyl alcohol titers as well as the overall effect on the cumulative yield and productivity. When the GCY1 gene is deleted the cumulative yield increased from 7.0% to 7.5%, and the productivity increased from 0.347 to 0.432 g/l/h in a 7 day fermentation which is a 7% and 25% increase in performance (FIG. 3), respectively for N=2 of each condition. When the promoter of the GCY1 gene is replaced with the pG2MAL promoter the cumulative yield increased from 7.0% to 7.3%, and the productivity increased from 0.347 to 0.367 g/l/h in a 7 day fermentation which is a 4% and 6% increase in performance (FIG. 3), respectively for N=2 of each condition.

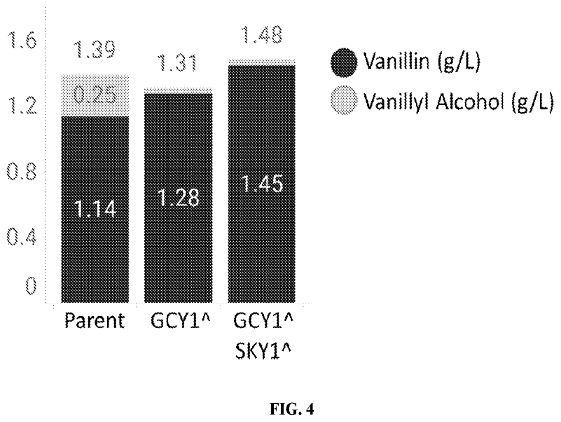

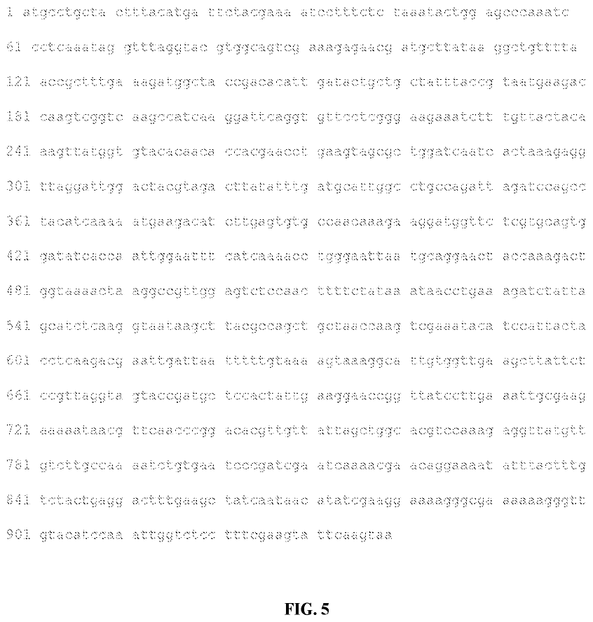

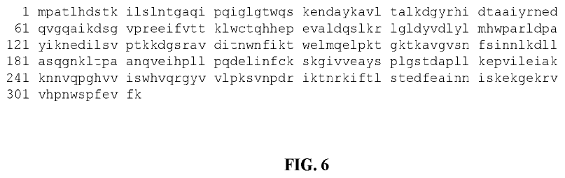

While the overall yield and productivity is only moderately improved over the parent, the amount of vanillyl alcohol produced in the mutant strain is significantly less than is produced in the parent strain. In the same fermentations described above, after 7 days of fermentation, the parent strain accumulates 3.5 g/L vanillyl alcohol while the mutant strain with the GCYI deleted decreases vanillyl alcohol to 0.73 g/L. The mutant strain where the promoter of the GCY1 gene is replaced with the pG2MAL promoter decreases vanillyl alcohol to 0.75 g/L vanillyl alcohol. This is a 4.8 fold and 4.7 fold reduction in vanillyl alcohol accumulation (FIG. 4), respectively for N=2 of each condition.

### Example 7. Combining the Deletion of the Native GCY1 Protein and the Native SKY1 Protein Increases the Amount of Total Vanillin Produced

When the native yeast genes GCY1 (YOR120W) and SKY1 (YMR216C) were deleted in strains producing vanillin and glucovanillin, there was a decrease in the amount of the undesired byproducts vanillyl alcohol and gluco-vanillyl alcohol. In addition, there was an increase in the amount of glucovanillin and vanillin (total vanillin) produced. Because all samples are first treated with a commercially available beta-glucosidase before measurement, only vanillin and vanillyl alcohol were directly measured. In all aspects, the amount of vanillyl alcohol produced by the GCY1\hat{ } and GCY1\hat{ }SKY1\hat{ } strains in 96-well plates decreased to quantities that are below the linear range for the assay to measure vanillyl alcohol (less than 0.12 g/L).

In one aspect, the entire ORF (open reading frame) of GCY1 (YOR120W) was deleted (FIG. 4) and the amount of vanillyl alcohol produced by the mutant strain decreased from 0.25 g/L to almost none while the amount of vanillin produced increased from 1.14 g/L to 1.29 g/L, a 13% improvement in vanillin production. In another aspect, the entire ORF (open reading frame) of GCY1 (YOR120W) and the entire ORF (open reading frame) of SKY1 (YMR216C) were both deleted and the amount of vanillyl alcohol produced by the mutant strain decreased from 0.25 g/L to almost none while the amount of vanillin produced increased from 1.14 g/L to 1.45 g/L, a 27% improvement in vanillin produced when compared to the parent strain or a 12% improvement over the strains with only the ORF of GCY1 (YOR120W) deleted.

FIG. 4 is a graph providing titers of g/L vanillin and g/L vanillyl alcohol for a 96-well plate experiment using a vanillin producing strain with the GCY1 and SKY1 intact (Parent), the ORF of the GCY1 deleted (GCY1\hat{ }), or the ORF of the GCY1 deleted and the ORF of the SKY1 deleted (GCY1\hat{ } SKY1\hat{ }). In all cases, samples were treated with a commercially available beta-glucosidase to convert glucovanillin into vanillin and gluco-vanillyl alcohol into vanillyl alcohol before quantification. Note that in strains labeled GCY1\hat{ } and GCY1\hat{ }SKY1\hat{ } the amount of vanillyl alcohol produced was below the linear range of the assay (less than 0.12 g/L).

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A genetically modified host cell capable of producing vanillin or glucovanillin, the host cell comprising one or more heterologous nucleic acids, each nucleic acid independently encoding at least one enzyme of a vanillin biosynthetic pathway, wherein native GCY1 activity is modified.

2. The genetically modified host cell of claim 1, wherein ORF of GCY (YOR120W) is modified.

3. The genetically modified host cell of claim 1, wherein the ORF of GCY1 is deleted.

4. The genetically modified host cell of claim 1, wherein the ORF of GCY 1 comprises the mutation A35T.

5. The genetically modified host cell of claim 1, wherein the GCY1 promoter is replaced with a pG2MAL or a pMAL32 promoter.

6. The genetically modified host cell of claim 1, wherein the GCY1 gene product comprises an amino acid sequence at least 80, 85, 90, 95, 99. or 100% identical to the GCY1 amino acid sequence encoded by nucleotides 1-939 of SEQ ID NO: 1.

7. The genetically modified host cell of any of claims 1-5, wherein the one or more heterologous nucleic acids are integrated into the genome of the host cell.

8. The genetically modified host cell of any of claims 1-5, wherein the one or more heterologous nucleic acids are selected from a group consisting of one or more copies of AroF, AroB, AroD, AroZ, OMT, ACAR, PPTase, UGT, and EAO.

9. The genetically modified host cell of claim 8, wherein the host cell encodes AroF, AroB, AroD, AroZ, OMT, ACAR, PPTase, UGT, and EAO.

10. The genetically modified host cell of claim 8, comprising two or more chromosomally integrated copies of AroZ, UGT, and OMT.

11. The genetically modified host cell of any of the previous claims comprising one or more nucleic acids expressing E. coli AroB, E. coli AroD, E. coli AroF, and Podospora pauciseta AroZ.

12. The genetically modified host cell of any of the previous claims further comprising one or more nucleic acids expressing Cornybacterium glutamicum PPTASE and Nocardia iowensis ACAR.

13. The genetically modified host cell of any of the previous claims further comprising one or more nucleic acids expressing Rhodococcus jostii EAO.

14. The genetically modified host cell of any of the previous claims further comprising one or more nucleic acids expressing Arabidopsis thaliana UGT.

15. The genetically modified host cell of any of claims 1-14, which has been genetically modified to delete one or more native genes selected from a group consisting of HFD1, EXG1, and SKY1.

16. The genetically modified host cell of any of the preceding claims, wherein each heterologous gene is expressed from an inducible promoter.

17. The genetically modified host cell of any of the preceding claims, wherein each heterologous gene is expressed from a GAL promoter.

18. The genetically modified host cell of any of the preceding claims, wherein each heterologous gene is expressed from a GAL promoter.

19. The genetically modified host cell of any of the preceding claims, wherein each heterologous gene is expressed from a GAL promoter, and wherein a GAL80 gene is expressed from a maltose-responsive promoter.

20. The genetically modified host cell of any of the preceding claims, wherein the host cell is selected from a bacterial cell, a fungal cell, an algal cell, an insect cell, and a plant cell.

21. The genetically modified host cell of any of the preceding claims, wherein the host cell is a yeast cell.

22. The genetically modified host cell of any of the preceding claims, wherein the host cell is a Saccharomyces cerevisiae cell.

23. The genetically modified host cell of any of the preceding claims, wherein the vanillin is vanillin or a glucovanillin.

24. The genetically modified host cell of any of the preceding claims, wherein the vanillin is a glucovanillin.

25. The genetically modified host cell of any of the previous claims that produces at least a 5, 10, 15, or 20% increase in peak cumulative yield or productivity, or both, compared to a parent strain. 26 A method of producing vanillin or glucovanillin, the method comprising:
providing a population of genetically modified host cells according to any of claims 1-25, and culturing said cells under conditions that promote the synthesis of vanillin or glucovanillin.

27. The method of claim 24, further comprising: prior to the culturing, growing the population of genetically modified host cells in a growth medium comprising a small molecule, wherein expression of at least one of the one or more heterologous nucleic acids is positively regulated by the activity of a promoter responsive to the small molecule, wherein the concentration of the small molecule in the growth medium is sufficient to repress the promoter, and wherein the concentration of the small molecule in the culture medium during the culturing is sufficiently low that the promoter is activated.

28. The method of claim 27, wherein the small molecule is maltose or lysine.

29. Vanillin or glucovanillin produced by the method of any of claims 26-28.

30. The genetically modified host cell of claim 14, wherein the Arabidopsis thaliana UGT is UGT72E2.

31. The genetically modified host cell of claim 19, wherein the maltose-responsive promoter is pG2MAL or pMAL32.


SEQUENCE LISTING (31 sequences)

SEQ ID NO: 1 - DNA, 939 bp, Saccharomyces cerevisiae
atgcctgctactttacatgattctacgaaaatcctttctctaaatactggagcccaaatccctcaaataggtttaggtacgtggcagtcgaaagagaacgatgcttataaggctgttttaaccgctttgaaagatggctaccgacacattgatactgctgctatttaccgtaatgaagaccaagtcggtcaagccatcaaggattcaggtgttcctcgggaagaaatctttgttactacaaagttatggtgtacacaacaccacgaacctgaagtagcgctggatcaatcactaaagaggttaggattggactacgtagacttatatttgatgcattggcctgccagattagatccagcctacatcaaaaatgaagacatcttgagtgtgccaacaaagaaggatggttctcgtgcagtggatatcaccaattggaatttcatcaaaacctgggaattaatgcaggaactaccaaagactggtaaaactaaggccgttggagtctccaacttttctataaataacctgaaagatctattagcatctcaaggtaataagcttacgccagctgctaaccaagtcgaaatacatccattactacctcaagacgaattgattaatttttgtaaaagtaaaggcattgtggttgaagcttattctccgttaggtagtaccgatgctccactattgaaggaaccggttatccttgaaattgcgaagaaaaataacgttcaacccggacacgttgttattagctggcacgtccaaagaggttatgttgtcttgccaaaatctgtgaatcccgatcgaatcaaaacgaacaggaaaatatttactttgtctactgaggactttgaagctatcaataacatatcgaaggaaaagggcgaaaaaagggttgtacatccaaattggtctcctttcgaagtattcaagtaa

SEQ ID NO: 2 - protein, 312 aa, Saccharomyces cerevisiae
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 3 - DNA, 711 bp, Artificial Sequence
  Synthetic pG2MAL_v1
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattgaatggcttaagtaggttgcaatttctttttctattagtagctaaaaatgggtcacgtgatctatattcgaaaggggcggttgcctcaggaaggcacagaaatattatctaaaagcgagagtttaagcgagttgcaagattcaggggtccatgtgccttggacgatattaaggcagaaggcagtatgtccgcgaaaatttccggataaatcagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 4 - DNA, 711 bp, Artificial Sequence
  Synthetic pG2MAL_v2
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattgaatggcttaagtaggttgcaatttctttttctattagtagctaaaaatgggtcacgtgatctatattcgaaaggggcggttgcctcaggaaggcacgtccgcgaaaatttccggataaatcttcaggggtccatgtgccttggacgatattaaggcagaaggcagtatagaaatattatctaaaagcgagagtttaagcgagttgcaagaagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 5 - DNA, 778 bp, Artificial Sequence
  Synthetic pG2MAL_v3
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaatttcttgcaactcgcttaaactctcgcttttagataatatttctgaatggcttaagtaggttgcaatttctttttctattagtagctaaaaatgggtcacgtgatctatattcgaaaggggcggttgcctcaggaaggcacgtccgcgaaaatttccggataaatcttcaggggtccatgtgccttggacgatattaaggcagaaggcagtatagaaatattatctaaaagcgagagtttaagcgagttgcaagaagattagttaagcccttcccatctcaagatggggagcaaatggcattatactcctgctaggatttatccggaaattttcgcggacaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 6 - DNA, 778 bp, Artificial Sequence
  Synthetic pG2MAL_v5
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaatttcttgcaactcgcttaaactctcgcttttagataatatttctgaatggcttaagtaggttgcaatttctttttctattagtagctaaaaatgggtcacgtgatctatattcgaaaggggcggttgcctcaggaaggcacgtccgcgaaaatttccggataaatcttcaggggtccatgtgccttggacgatattaaggcagaaggcagtatgatttatccggaaattttcgcggacagattagttaagcccttcccatctcaagatggggagcaaatggcattatactcctgctagagaaatattatctaaaagcgagagtttaagcgagttgcaagaaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 7 - DNA, 794 bp, Artificial Sequence
  Synthetic pG2MAL_v6
aataccagtttcgctgcagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattgaatggcttaagtaggttgcaatttctttttctattagtagctaaaaatgggtcacgtgatctatattcgaaaggggcggttgcctcaggaaggcacttcaggggtccatgtgccttggacgatattaaggcagaaggcagtattcttgcaactcgcttaaactctcgcttttagataatatttctagattagttaagcccttcccagtccgcgaaaatttccggataaatctctcaagatggggagcaaatggcattatactcctgctaggatttatccggaaattttcgcggacaaagttaactgtgcacatattcttaaattatacaatgttctggagagctattgtttaaaaaacaaacatttcgcagaaatattatctaaaagcgagagtttaagcgagttgcaagaaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 8 - DNA, 778 bp, Artificial Sequence
  Synthetic pG2MAL_v7
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattagaaatattatctaaaagcgagagtttaagcgagttgcaagagaatggcttaagtaggttgcaatttctttttctattagtagctaaaaatgggtcacgtgatctatattcgaaaggggcggttgcctcaggaaggcacgtccgcgaaaatttccggataaatcttcaggggtccatgtgccttggacgatattaaggcagaaggcagtatagaaatattatctaaaagcgagagtttaagcgagttgcaagaagattagttaagcccttcccatctgtccgcgaaaatttccggataaatccaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 9 - DNA, 845 bp, Artificial Sequence
  Synthetic pG2MAL_v8
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattgtccgcgaaaatttccggataaatcgtccgcgaaaatttccggataaatcgtccgcgaaaatttccggataaatcgaatggcttaagtaggttgcaatttctttttctattagtagctaaaaatgggtcacgtgatctatattcgaaaggggcggttgcctcaggaaggcacttcaggggtccatgtgccttggacgatattaaggcagaaggcagtatagaaatattatctaaaagcgagagtttaagcgagttgcaagaagaaatattatctaaaagcgagagtttaagcgagttgcaagaagaaatattatctaaaagcgagagtttaagcgagttgcaagaagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 10 - DNA, 778 bp, Artificial Sequence
  Synthetic pG2MAL_v9
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattgtccgcgaaaatttccggataaatcgaatggcttaagtaggttgcaatttctttttctattagtagctaaaaatgggtcacgtgatctataagaaatattatctaaaagcgagagtttaagcgagttgcaagattcgaaaggggcggttgcctcaggaaggcacgtccgcgaaaatttccggataaatcttcaggggtccatgtgccttggacgatattaaggcagaaggcagtatagaaatattatctaaaagcgagagtttaagcgagttgcaagaagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 11 - DNA, 845 bp, Artificial Sequence
  Synthetic pG2MAL_v10
cagaaggcacatctattacatttactgagcataacgggctgtactaatccaaggaggtttacggaccagaggaactttccagattcagatcacagcaatataggactggaaaacatcaggtagccgcactcaacttgtaactggcaactactttgcattaaactccaattaaatgcggtagaatcttttcagaaaaggtattcaacgtcaattagaaatattatctaaaagcgagagtttaagcgagttgcaagagaatggcttaagtaggttgcaatttctttttctattagtagctaaaaatgggtcacgtgatctatagtccgcgaaaatttccggataaatcttcgaaaggggcggttgcctcaggaaggcacagaaatattatctaaaagcgagagtttaagcgagttgcaagattcaggggtccatgtgccttggacgatattaaggcagaaggcagtatgtccgcgaaaatttccggataaatcagattagttaagcccttccagaaatattatctaaaagcgagagtttaagcgagttgcaagacatctcaagatggggagcaaatggcattatactcctgctagaaagttaactgtgcgtccgcgaaaatttccggataaatcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacat

SEQ ID NO: 12 - DNA, 875 bp, Artificial Sequence
  Synthetic pMAL32
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa

SEQ ID NO: 13 - DNA, 808 bp, Artificial Sequence
  Synthetic pMAL32_v1
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagatccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatca

SEQ ID NO: 14 - DNA, 1253 bp, Artificial Sequence
  Synthetic pMAL1
gatgatggacactagtgtgtcgagaatgtatcaactatatatagtcctaatgccacacaaatatgaagtgggggaagcccattcttaatccggctcaattttggtgcgtgatcgcggcctatgtttgcttccagaaaaagcttagaataatatttctcacctttgatggaatgctcgcgagtgctcgttttgattaccccatatgcattgttgcagcatgcaagcactattgcaagccacgcatggaagaaatttgcaaacacctatagccccgcgttgttgaggaggtggacttggtgtaggaccataaagctgtgcactactatggtgagctctgtcgtctggtgaccttctatctcaggcacatcctcgtttttgtgcatgaggttcgagtcacgcccacggcctattaatccgcgaaataaatgcgaaatctaaattatgacgcaaggctgagagattctgacacgccgcatttgcggggcagtaattatcgggcagttttccggggttcgggatggggtttggagagaaagttcaacacagaccaaaacagcttgggaccacttggatggaggtccccgcagaagagctctggcgcgttggacaaacattgacaatccacggcaaaattgtctacagttccgtgtatgcggatagggatatcttcgggagtatcgcaataggatacaggcactgtgcagattacgcgacatgatagctttgtatgttctacagactctgccgtagcagtctagatataatatcggagttttgtagcgtcgtaaggaaaacttgggttacacaggtttcttgagagccctttgacgttgattgctctggcttccatccaggccctcatgtggttcaggtgcctccgcagtggctggcaagcgtgggggtcaattacgtcacttctattcatgtaccccagactcaattgttgacagcaatttcagcgagaattaaattccacaatcaattctcgctgaaataattaggccgtgatttaattctcgctgaaacagaatcctgtctggggtacagataacaatcaagtaactattatggacgtgcataggaggtggagtccatgacgcaaagggaaatattcattttatcctcgcgaagttgggatgtgtcaaagcgtcgcgctcgctatagtgatgagaatgtctttagtaagcttaagccatataaagaccttccgcctccatatttttttttatccctcttgacaatattaattcctt

SEQ ID NO: 15 - DNA, 1253 bp, Artificial Sequence
  Synthetic pMAL2
aaggaattaatattgtcaagagggataaaaaaaaatatggaggcggaaggtctttatatggcttaagcttactaaagacattctcatcactatagcgagcgcgacgctttgacacatcccaacttcgcgaggataaaatgaatatttccctttgcgtcatggactccacctcctatgcacgtccataatagttacttgattgttatctgtaccccagacaggattctgtttcagcgagaattaaatcacggcctaattatttcagcgagaattgattgtggaatttaattctcgctgaaattgctgtcaacaattgagtctggggtacatgaatagaagtgacgtaattgacccccacgcttgccagccactgcggaggcacctgaaccacatgagggcctggatggaagccagagcaatcaacgtcaaagggctctcaagaaacctgtgtaacccaagttttccttacgacgctacaaaactccgatattatatctagactgctacggcagagtctgtagaacatacaaagctatcatgtcgcgtaatctgcacagtgcctgtatcctattgcgatactcccgaagatatccctatccgcatacacggaactgtagacaattttgccgtggattgtcaatgtttgtccaacgcgccagagctcttctgcggggacctccatccaagtggtcccaagctgttttggtctgtgttgaactttctctccaaaccccatcccgaaccccggaaaactgcccgataattactgccccgcaaatgcggcgtgtcagaatctctcagccttgcgtcataatttagatttcgcatttatttcgcggattaataggccgtgggcgtgactcgaacctcatgcacaaaaacgaggatgtgcctgagatagaaggtcaccagacgacagagctcaccatagtagtgcacagctttatggtcctacaccaagtccacctcctcaacaacgcggggctataggtgtttgcaaatttcttccatgcgtggcttgcaatagtgcttgcatgctgcaacaatgcatatggggtaatcaaaacgagcactcgcgagcattccatcaaaggtgagaaatattattctaagctttttctggaagcaaacataggccgcgatcacgcaccaaaattgagccggattaagaatgggcttcccccacttcatatttgtgtggcattaggactatatatagttgatacattctcgacacactagtgtccatcatc

SEQ ID NO: 16 - DNA, 785 bp, Artificial Sequence
  Synthetic pMAL11
ttatgtaatttagttacgcttgactgatgtacatttgagattatcaaaaaaactgcttaagagatggatgatttaattttttagagacgtattaatggaactttttataccttgcccagagcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataat

SEQ ID NO: 17 - DNA, 785 bp, Artificial Sequence
  Synthetic pMAL12
attatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataa

SEQ ID NO: 18 - DNA, 875 bp, Artificial Sequence
  Synthetic pMAL31
ttatgtattttagttacgcttgactgatgtacatttgagattatcaaaaaaactgcttaagagatagatggtttaattttttagagacgtattaatggaactttttataccttgcccagagcgcctcaagaaaatgatgctgaaagaagaattgaggaaggaactactcatcttacgttgtttgtatcatcccacgatccaaatcatgttacctacgttaggtacgctaggaactgaaaaaagaaaagaaaagtatgcgttatcactcttcgagccaattcttaattgtgtggggtccgcgaaaacttccggataaatcctgtaaacttaaacttaaaccccgtgtttagcgaaattttcaacgaagcgcgcaataaggagaaatattatataaaagcgagagtttaagcgaggttgcaagaatctctacggtacagatgcaacttactatagccaaggtctattcgtattggtatccaagcagtgaagctactcaggggaaaacatattttcagagatcaaagttatgtcagtctctttttcatgtgtaacttaacgtttgtgcaggtatcataccggcctccacataatttttgtggggaagacgttgttgtagcagtctccttatactctccaacaggtgtttaaagacttcttcaggcctcatagtctacatctggagacaacattagatagaagtttccacagaggcagctttcaatatactttcggctgtgtacatttcatcctgagtgagcgcatattgcataagtactcagtatataaagagacacaatatactccatacttgttgtgagtggttttagcgtattcagtataacaataagaattacatccaagactattaattaact

SEQ ID NO: 19 - DNA, 643 bp, Artificial Sequence
  Synthetic pGMAL_v5
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtcttgcaacctcgcttaaactctcgcttttatataatatttcttccgtgcgtcctggtcttcaccggtcgcgttcctgaaacgcagatgtgcctaacaataaagatttatccggaagttttcgcggacgattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 20 - DNA, 709 bp, Artificial Sequence
  Synthetic pGMAL_v6
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagagaaatattatctaaaagcgagagtttaagcgagttgcaagaagaaatattatctaaaagcgagagtttaagcgagttgcaagatccgtgcgtcctggtcttcaccggtcgcgttcctgaaacgcagatgtgcctaacaataaagtccgcgaaaatttccggataaatcgtccgcgaaaatttccggataaatcgattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 21 - DNA, 709 bp, Artificial Sequence
  Synthetic pGMAL_v7
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagagaaatattatctaaaagcgagagtttaagcgagttgcaagatcttgcaactcgcttaaactctcgcttttagataatatttcttccgtgcgtcctggtcttcaccggtcgcgttcctgaaacgcagatgtgcctaacaataaagtccgcgaaaatttccggataaatcgatttatccggaaattttcgcggacgattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 22 - DNA, 776 bp, Artificial Sequence
  Synthetic pGMAL_v9
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagagaaatattatctaaaagcgagagtttaagcgagttgcaagaagaaatattatctaaaagcgagagtttaagcgagttgcaagaagaaatattatctaaaagcgagagtttaagcgagttgcaagatccgtgcgtcctggtcttcaccggtcgcgttcctgaaacgcagatgtgcctaacaataaagtccgcgaaaatttccggataaatcgtccgcgaaaatttccggataaatcgtccgcgaaaatttccggataaatcgattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 23 - DNA, 843 bp, Artificial Sequence
  Synthetic pGMAL_v10
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagagaaatattatctaaaagcgagagtttaagcgagttgcaagaagaaatattatctaaaagcgagagtttaagcgagttgcaagaagaaatattatctaaaagcgagagtttaagcgagttgcaagaagaaatattatctaaaagcgagagtttaagcgagttgcaagatccgtgcgtcctggtcttcaccggtcgcgttcctgaaacgcagatgtgcctaacaataaagtccgcgaaaatttccggataaatcgtccgcgaaaatttccggataaatcgtccgcgaaaatttccggataaatcgtccgcgaaaatttccggataaatcgattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 24 - DNA, 776 bp, Artificial Sequence
  Synthetic pGMAL_v11
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaaggtccgcgaaaatttccggataaatcgtccgcgaaaatttccggataaatcgtccgcgaaaatttccggataaatctccgtgcgtcctggtcttcaccggtcgcgttcctgaaacgcagatgtgcctaacaataaaagaaatattatctaaaagcgagagtttaagcgagttgcaagaagaaatattatctaaaagcgagagtttaagcgagttgcaagaagaaatattatctaaaagcgagagtttaagcgagttgcaagagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 25 - DNA, 843 bp, Artificial Sequence
  Synthetic pGMAL_v12
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaaggtccgcgaaaatttccggataaatcgtccgcgaaaatttccggataaatcgtccgcgaaaatttccggataaatcgtccgcgaaaatttccggataaatctccgtgcgtcctggtcttcaccggtcgcgttcctgaaacgcagatgtgcctaacaataaaagaaatattatctaaaagcgagagtttaagcgagttgcaagaagaaatattatctaaaagcgagagtttaagcgagttgcaagaagaaatattatctaaaagcgagagtttaagcgagttgcaagaagaaatattatctaaaagcgagagtttaagcgagttgcaagagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 26 - DNA, 776 bp, Artificial Sequence
  Synthetic pGMAL_v13
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaacctagaaatattatctaaaagcgagagtttaagcgagttgcaagatctctttggaactttcagtaatacgcttgtccgcgaaaatttccggataaatcaactgctcattgctatattgaagtccgtgcgtcctggtcttcaccggtcgcgttcctgaaacgcagatgtgcctaacaataaagattctaagaaatattatctaaaagcgagagtttaagcgagttgcaagacaatactagcttttatggttatgagtccgcgaaaatttccggataaatcagaggaaaaattggcagtaaccagaaatattatctaaaagcgagagtttaagcgagttgcaagatggccccacaaaccttcaaatcaacgaatcaaattaacgtccgcgaaaatttccggataaatcaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 27 - DNA, 708 bp, Artificial Sequence
  Synthetic pGMAL_v14
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaacctagaaatattatctaaaagcgagagtttaagcgagttgcaagatctctttggaactttcagtaatacgcttgtccgcgaaaatttccggataaatcaactgctcattgctatattgaagtccgtgcgtcctggtcttcaccggtcgcgttcctgaaacgcagatgtgcctaacaataaagattctaagaaatattatctaaaagcgagagtttaagcgagttgcaagacaatactagcttttatggttatgagtccgcgaaaatttccggataaatcagaggaaaaattggcagtaacctggccccacaaaccttcaaatcaacgaatcaaattaaaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 28 - DNA, 709 bp, Artificial Sequence
  Synthetic pGMAL_v15
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtccgtgcgtcctggtcttcaccggtcgcgttcctgaaacgcagatgtgcctaacaataaagattctaagaaatattatctaaaagcgagagtttaagcgagttgcaagacaatactagcttttatggttatgagtccgcgaaaatttccggataaatcagaggaaaaattggcagtaaccagaaatattatctaaaagcgagagtttaagcgagttgcaagatggccccacaaaccttcaaatcaacgaatcaaattaacgtccgcgaaaatttccggataaatcaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 29 - DNA, 843 bp, Artificial Sequence
  Synthetic pGMAL_v16
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtccgtgcgtcctggtcttcaccggtcgcgttcctgaaacgcagatgtgcctaacaataaagattctacaatactagcttttatggttatgaagaggaaaaattggcagtaacctggccccacaaaccttcaaatcaacgaatcaaattaacagaaatattatctaaaagcgagagtttaagcgagttgcaagaagaaatattatctaaaagcgagagtttaagcgagttgcaagaagaaatattatctaaaagcgagagtttaagcgagttgcaagaagaaatattatctaaaagcgagagtttaagcgagttgcaagaaaccataggataataatgcgattagtttgtccgcgaaaatttccggataaatcgtccgcgaaaatttccggataaatcgtccgcgaaaatttccggataaatcgtccgcgaaaatttccggataaatctttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 30 - DNA, 776 bp, Artificial Sequence
  Synthetic pGMAL_v17
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtccgtgcgtcctggtcttcaccggtcgcgttcctgaaacgcagatgtgcctaacaataaagattctacaatactagcttttatggttatgaagaggaaaaattggcagtaacctggccccacaaaccttcaaatcaacgaatcaaattaacagaaatattatctaaaagcgagagtttaagcgagttgcaagaagaaatattatctaaaagcgagagtttaagcgagttgcaagaagaaatattatctaaaagcgagagtttaagcgagttgcaagaaaccataggataataatgcgattagtttgtccgcgaaaatttccggataaatcgtccgcgaaaatttccggataaatcgtccgcgaaaatttccggataaatctttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt

SEQ ID NO: 31 - DNA, 709 bp, Artificial Sequence
  Synthetic pGMAL_v18
ttatattgaattttcaaaaattcttactttttttttggatggacgcaaagaagtttaataatcatattacatggcaataccaccatatacatatccatatctaatcttacttatatgttgtggaaatgtaaagagccccattatcttagcctaaaaaaaccttctctttggaactttcagtaatacgcttaactgctcattgctatattgaagtccgtgcgtcctggtcttcaccggtcgcgttcctgaaacgcagatgtgcctaacaataaagattctacaatactagcttttatggttatgaagaggaaaaattggcagtaacctggccccacaaaccttcaaatcaacgaatcaaattaacagaaatattatctaaaagcgagagtttaagcgagttgcaagaagaaatattatctaaaagcgagagtttaagcgagttgcaagaaaccataggataataatgcgattagtttgtccgcgaaaatttccggataaatcgtccgcgaaaatttccggataaatctttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactt